SEQUENCE LISTING (46 sequences)

SEQ ID NO: 1 - protein, 298 aa, Pneumovirus human respiratory syncytial virus
MSKTKDQRTAKTLEKTWDTLNHLLFISSCLYKLNLKSIAQITLSILAMIISTSLIIVAIIFIASANNKVTLTTAIIQDATSQIKNTTPTYLTQNPQLGISFFNLSGTISQTTAILALTTPSVESILQSTTVKTKNTTTTQIQPSKPTTKQRQNKPPNKPNDDFHFEVFNFVPCSICSNNPTCWAICKRIPSKKPGKKTTTKPTKKPTIKTTKKDLKPQTTKPKEAPTTKPTEKPTINITKPNIRTTLLTNSTTGNLEHTSQEETLHSTSSESSTSPSQIYTTSEYLSQPPSPSNITDQ

SEQ ID NO: 2 - protein, 574 aa, Pneumovirus human respiratory syncytial virus
MELPILKTNAITTILAAVTLCFASSQNITEEFYQSTCSAVSKGYLSALRTGWYTSVITIELSNIKENKCNGTDAKVKLIKQELDKYKNAVTELQLLMQSTPAANNRARRELPRFMNYTLNNTKNNNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKADVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNVGKSTTNIMITTIIIVIIVILLLLIAVGLFLYCKARSTPVTLSKDQLSGINNIAFSN

SEQ ID NO: 3 - protein, 40 aa, Artificial
  RSV G polypeptide
RQNKPPNKPNDDFHFEVFNFVPCSICSNNPTCWAICKRIP

SEQ ID NO: 4 - protein, 36 aa, Artificial
  RSV G polypeptide
RQNKPPNKPNDDFHFEVFNFVPCSICSNNPTCWAIC

SEQ ID NO: 5 - protein, 22 aa, Artificial
  RSV G polypeptide
RQNKPPNKPNDDFHFEVFNFVP

SEQ ID NO: 6 - protein, 86 aa, Artificial
  RSV F polypeptide
TLPSEVNLCNIDIFNPKYDCKIMTSKADVSSSVITSLGAIVSCYGKTKCTASNKNRGIIKTFSNGCDYVSNKGVDTVSVGNTLYYV

SEQ ID NO: 7 - protein, 78 aa, Artificial
  RSV F polypeptide
CNIDIFNPKYDCKIMTSKADVSSSVITSLGAIVSCYGKTKCTASNKNRGIIKTFSNGCDYVSNKGVDTVSVGNTLYYV

SEQ ID NO: 8 - protein, 71 aa, Artificial
  RSV F polypeptide
ITREFSVNAGVTTPVSTYMLTNSELLSLINDMPITNDQKKLMSNNVQIVRQQSYSIMSIIKEEVLAYVVQL

SEQ ID NO: 9 - protein, 53 aa, Artificial
  RSV F polypeptide
ITREFSVNAGVTTPVSTYMLTNSELLSLINDMPITNDQKKLMSNNVQIVRQQS

SEQ ID NO: 10 - DNA, 120 bp, Artificial
  RSV G polynucleotide
cgccaaaacaaaccaccaaacaaacccaatgatgattttcactttgaagtgttcaactttgtaccctgcagcatatgcagcaacaatccaacttgctgggccatctgcaaaagaatacca

SEQ ID NO: 11 - DNA, 120 bp, Artificial
  RSV G polynucleotide
aggcagaataagccacccaataagcctaacgacgacttccatttcgaggtctttaatttcgtgccttgttctatctgttctaataaccccacctgttgggctatttgtaagcgtatcccc

SEQ ID NO: 12 - DNA, 108 bp, Artificial
  RSV G polynucleotide
cgccaaaacaaaccaccaaacaaacccaatgatgattttcactttgaagtgttcaactttgtaccctgcagcatatgcagcaacaatccaacttgctgggccatctgc

SEQ ID NO: 13 - DNA, 108 bp, Artificial
  RSV G polynucleotide
aggcagaataagccacccaataagcctaacgacgacttccatttcgaggtctttaatttcgtgccttgttctatctgttctaataaccccacctgttgggctatttgt

SEQ ID NO: 14 - DNA, 66 bp, Artificial
  RSV G polynucleotide
cgccaaaacaaaccaccaaacaaacccaatgatgattttcactttgaagtgttcaactttgtaccc

SEQ ID NO: 15 - DNA, 66 bp, Artificial
  RSV G polynucleotide
aggcagaataagccacccaataagcctaacgacgacttccatttcgaggtctttaatttcgtgcct

SEQ ID NO: 16 - DNA, 234 bp, Artificial
  RSV F polynucleotide
tgcaacattgacatattcaaccctaaatatgattgcaaaattatgacttcaaaagcagatgtaagcagctccgttatcacatctctaggagccattgtgtcatgctatggaaaaactaaatgtacagcatccaataaaaatcgtggaatcataaagacattttctaacgggtgtgattatgtatcaaataagggggtggacactgtatctgtaggtaatacattatattatgta

SEQ ID NO: 17 - DNA, 234 bp, Artificial
  RSV F polynucleotide
tgtaacatcgacattttcaaccctaaatacgactgcaagattatgacttccaaggccgatgtgagctcgtccgtaatcacaagtctcggagcgatcgtcagttgctacggcaagactaagtgcaccgctagcaataagaatagaggcattataaagactttctctaacggttgtgactacgtgtcaaacaaaggcgtagacaccgtgtccgtcggtaacacactgtactacgtg

SEQ ID NO: 18 - DNA, 234 bp, Artificial
  RSV F polynucleotide
tgtaacatcgacatcttcaatcctaaatacgactgcaaaattatgacgagcaaggctgatgtgtcatcttcagtcataaccagtctgggtgctatcgtttcctgctatgggaagaccaaatgcaccgcatcaaacaaaaaccggggcattatcaaaacattttctaacggatgcgattacgtgtctaataagggggtcgacactgtatctgtggggaacaccctttactatgtg

SEQ ID NO: 19 - DNA, 234 bp, Artificial
  RSV F polynucleotide
tgcaacattgacatattcaaccctaaatatgattgcaaaattatgacttcaaaagcagatgtaagcagctccgttatcacatctctaggagccattgtgtcatgctatggaaaaactaaatgtacagcatccaataaaaatcgtggaatcataaagacattttctaacgggtccgattatgtatcaaataagggggtggacactgtatctgtaggtaatacattatattatgta

SEQ ID NO: 20 - DNA, 234 bp, Artificial
  RSV F polynucleotide
tgtaacatcgacattttcaaccctaaatacgactgcaagattatgacttccaaggccgatgtgagctcgtccgtaatcacaagtctcggagcgatcgtcagttgctacggcaagactaagtgcaccgctagcaataagaatagaggcattataaagactttctctaacggttccgactacgtgtcaaacaaaggcgtagacaccgtgtccgtcggtaacacactgtactacgtg

SEQ ID NO: 21 - DNA, 234 bp, Artificial
  RSV F polynucleotide
tgtaacatcgacatcttcaatcctaaatacgactgcaaaattatgacgagcaaggctgatgtgtcatcttcagtcataaccagtctgggtgctatcgtttcctgctatgggaagaccaaatgcaccgcatcaaacaaaaaccggggcattatcaaaacattttctaacggatccgattacgtgtctaataagggggtcgacactgtatctgtggggaacaccctttactatgtg

SEQ ID NO: 22 - DNA, 213 bp, Artificial
  RSV F polynucleotide
attaccagggaatttagtgttaatgcaggtgtaactacacctgtaagcacttatatgttaacaaatagtgaattattatcattaatcaatgatatgcctataacaaatgatcagaaaaagttaatgtccaacaatgttcaaatagttagacagcaaagttactctatcatgtccataataaaggaggaagtcttagcatatgtagtacaattg

SEQ ID NO: 23 - DNA, 213 bp, Artificial
  RSV F polynucleotide
atcacaagggaattttcagtcaacgccggagtcaccacacccgtgtccacatacatgcttactaactccgagttgctttctctgatcaacgacatgcctatcacaaacgaccagaagaagctgatgagcaacaacgttcagattgtccgtcaacagtcttactcgattatgtctattatcaaggaggaagtattggcatacgttgttcagctc

SEQ ID NO: 24 - DNA, 213 bp, Artificial
  RSV F polynucleotide
atcactcgggagttcagcgtgaatgcgggggtgacgacccctgtttcaacttacatgctgaccaattctgaattgctctccctcatcaacgacatgccaatcaccaatgaccagaaaaagctgatgagtaacaacgttcagattgttcgccaacagagctatagcatcatgagtatcattaaggaggaggtcctggcctatgttgtacagctc

SEQ ID NO: 25 - DNA, 159 bp, Artificial
  RSV F polynucleotide
attaccagggaatttagtgttaatgcaggtgtaactacacctgtaagcacttatatgttaacaaatagtgaattattatcattaatcaatgatatgcctataacaaatgatcagaaaaagttaatgtccaacaatgttcaaatagttagacagcaaagt

SEQ ID NO: 26 - DNA, 159 bp, Artificial
  RSV F polynucleotide
atcacaagggaattttcagtcaacgccggagtcaccacacccgtgtccacatacatgcttactaactccgagttgctttctctgatcaacgacatgcctatcacaaacgaccagaagaagctgatgagcaacaacgttcagattgtccgtcaacagtct

SEQ ID NO: 27 - DNA, 159 bp, Artificial
  RSV F polynucleotide
atcactcgggagttcagcgtgaatgcgggggtgacgacccctgtttcaacttacatgctgaccaattctgaattgctctccctcatcaacgacatgccaatcaccaatgaccagaaaaagctgatgagtaacaacgttcagattgttcgccaacagagc

SEQ ID NO: 28 - DNA, 258 bp, Artificial
  RSV F polynucleotide
acattaccaagtgaagtaaatctctgcaacattgacatattcaaccctaaatatgattgcaaaattatgacttcaaaagcagatgtaagcagctccgttatcacatctctaggagccattgtgtcatgctatggaaaaactaaatgtacagcatccaataaaaatcgtggaatcataaagacattttctaacgggtgtgattatgtatcaaataagggggtggacactgtatctgtaggtaatacattatattatgta

SEQ ID NO: 29 - DNA, 258 bp, Artificial
  RSV F polynucleotide
accttgccctcagaggtaaatttgtgtaacatcgacattttcaaccctaaatacgactgcaagattatgacttccaaggccgatgtgagctcgtccgtaatcacaagtctcggagcgatcgtcagttgctacggcaagactaagtgcaccgctagcaataagaatagaggcattataaagactttctctaacggttgtgactacgtgtcaaacaaaggcgtagacaccgtgtccgtcggtaacacactgtactacgtg

SEQ ID NO: 30 - DNA, 258 bp, Artificial
  RSV F polynucleotide
acactgcccagtgaggttaatctgtgtaacatcgacatcttcaatcctaaatacgactgcaaaattatgacgagcaaggctgatgtgtcatcttcagtcataaccagtctgggtgctatcgtttcctgctatgggaagaccaaatgcaccgcatcaaacaaaaaccggggcattatcaaaacattttctaacggatgcgattacgtgtctaataagggggtcgacactgtatctgtggggaacaccctttactatgtg

SEQ ID NO: 31 - DNA, 258 bp, Artificial
  RSV F polynucleotide
acattaccaagtgaagtaaatctctgcaacattgacatattcaaccctaaatatgattgcaaaattatgacttcaaaagcagatgtaagcagctccgttatcacatctctaggagccattgtgtcatgctatggaaaaactaaatgtacagcatccaataaaaatcgtggaatcataaagacattttctaacgggtccgattatgtatcaaataagggggtggacactgtatctgtaggtaatacattatattatgta

SEQ ID NO: 32 - DNA, 258 bp, Artificial
  RSV F polynucleotide
accttgccctcagaggtaaatttgtgtaacatcgacattttcaaccctaaatacgactgcaagattatgacttccaaggccgatgtgagctcgtccgtaatcacaagtctcggagcgatcgtcagttgctacggcaagactaagtgcaccgctagcaataagaatagaggcattataaagactttctctaacggttccgactacgtgtcaaacaaaggcgtagacaccgtgtccgtcggtaacacactgtactacgtg

SEQ ID NO: 33 - DNA, 258 bp, Artificial
  RSV F polynucleotide
acactgcccagtgaggttaatctgtgtaacatcgacatcttcaatcctaaatacgactgcaaaattatgacgagcaaggctgatgtgtcatcttcagtcataaccagtctgggtgctatcgtttcctgctatgggaagaccaaatgcaccgcatcaaacaaaaaccggggcattatcaaaacattttctaacggatccgattacgtgtctaataagggggtcgacactgtatctgtggggaacaccctttactatgtg

SEQ ID NO: 34 - DNA, 894 bp, Pneumovirus human respiratory syncytial virus
atgtccaaaaccaaggaccaacgcaccgccaagacactagaaaagacctgggacactctcaatcatctattattcatatcatcgtgcttatacaagttaaatcttaaatctatagcacaaatcacattatccattctggcaatgataatctcaacttcacttataattgtagctatcatattcatagcctcagcaaacaacaaagtcacactaacaactgcaatcatacaagatgcaacaagccagatcaagaacacaaccccaacatacctgacccagaatccccagcttggaatcagcttcttcaatctgtctggaactatatcacaaaccaccgccatactagctttaacaacaccaagtgtcgagtcaatcctgcaatctacaacagtcaagaccaaaaacacaacaacaacccaaatacaacccagcaagcccaccacaaaacaacgccaaaacaaaccaccaaacaaacccaatgatgattttcactttgaagtgttcaactttgtaccctgcagcatatgcagcaacaatccaacttgctgggccatctgcaaaagaataccaagcaaaaaacctggaaagaaaaccaccaccaagcccacgaaaaaaccaaccatcaagacaaccaaaaaagatctcaaacctcaaaccacaaaaccaaaagaagcacctaccaccaagcccacagaaaagccaaccatcaacatcaccaaaccaaacatcagaactacactgctcaccaacagtaccacaggaaatctagaacacacaagtcaagaggaaaccctccattcaacctcctccgaaagcagtacaagcccttcacaaatctatacaacatccgagtacctatcacaacctccatctccatccaacataacagaccag

SEQ ID NO: 35 - DNA, 894 bp, Artificial
  insect cell codon optimized polynucleotide RSV G
atgagtaagactaaagatcagaggactgctaaaaccttggagaaaacttgggataccttgaaccacttgctgtttatcagcagctgtctgtataaactgaacttgaagagcatcgctcagattaccctgagtatcctcgctatgatcattagcaccagcttgatcatcgtggccattatctttatcgctagcgctaataacaaggtgaccttgaccaccgctattatccaggacgctacctctcaaatcaagaacactactcccacctatctcactcaaaaccctcaattgggtatttcgtttttcaacctcagcggtaccatcagccagactactgctatcttggccctgaccaccccctccgtggaaagcattctccagagcaccaccgtgaaaactaagaataccaccaccactcagatccagccttcaaaacctacaaccaagcagaggcagaataagccacccaataagcctaacgacgacttccatttcgaggtctttaatttcgtgccttgttctatctgttctaataaccccacctgttgggctatttgtaagcgtatcccctcaaagaagccgggtaaaaagactactacgaaaccaactaagaagcccacgattaaaaccactaagaaggacttgaagccccagactaccaagcccaaggaggctcccactactaaacctaccgagaaacccaccattaacatcactaagcccaatattcgtaccaccctcttgactaattccactaccggtaacttggagcatacctcccaggaagagactttgcacagcactagtagtgagtcttccacctctcccagccaaatctacaccaccagtgaatatttgagccagccacccagccccagtaatatcaccgatcaa

SEQ ID NO: 36 - DNA, 1722 bp, Pneumovirus human respiratory syncytial virus
atggagttgccaatcctcaaaacaaatgcaattaccacaatccttgctgcagtcacactctgtttcgcttccagtcaaaacatcactgaagaattttatcaatcaacatgcagtgcagttagcaaaggctatcttagtgctttaagaactggttggtatactagtgttataactatagaattaagtaatatcaaggaaaataagtgtaatggaacagacgctaaggtaaaattgataaaacaagaattagataaatataaaaatgctgtaacagaattgcagttgctcatgcaaagcacaccagcagccaacaatcgagccagaagagaactaccaaggtttatgaattatacactcaacaataccaaaaataacaatgtaacattaagcaagaaaaggaaaagaagatttcttggctttttgttaggtgtaggatctgcaatcgccagtggcattgctgtatctaaagtcctgcacctagaaggggaagtgaacaaaatcaaaagtgctctactatccacaaacaaggctgtagtcagcttatcaaatggagttagtgtcttaaccagcaaagtgttagacctcaaaaactatatagataaacagttgttacctattgtgaacaagcaaagctgcagcatatcaaacattgaaactgtgatagaattccaacaaaagaacaacagactactagagattaccagggaatttagtgttaatgcaggtgtaactacacctgtaagcacttatatgttaacaaatagtgaattattatcattaatcaatgatatgcctataacaaatgatcagaaaaagttaatgtccaacaatgttcaaatagttagacagcaaagttactctatcatgtccataataaaggaggaagtcttagcatatgtagtacaattgccactatatggtgtaatagatacaccttgttggaaactacatacatcccctctatgcacaaccaacacaaaggaagggtccaacatctgtttaacaagaaccgacagaggatggtactgtgacaatgcaggatcagtgtctttcttcccacaagctgaaacatgcaaagttcaatcgaatcgagtattttgtgacacaatgaacagtttaacattaccaagtgaagtaaatctctgcaacattgacatattcaaccctaaatatgattgcaaaattatgacttcaaaagcagatgtaagcagctccgttatcacatctctaggagccattgtgtcatgctatggaaaaactaaatgtacagcatccaataaaaatcgtggaatcataaagacattttctaacgggtgtgattatgtatcaaataagggggtggacactgtatctgtaggtaatacattatattatgtaaataagcaagaaggaaaaagtctctatgtaaaaggtgaaccaataataaatttctatgacccattagtgttcccttctgatgaatttgatgcatcaatatctcaagtcaatgagaagattaaccagagcctagcatttattcgtaaatccgatgaattattacataatgtaaatgttggtaaatccaccacaaatatcatgataactactataattatagtgattatagtaatattgttattattaattgcagttgggttgttcctatactgcaaggccagaagcacaccagtcacactaagcaaggatcaactgagtggtataaataatattgcatttagtaac

SEQ ID NO: 37 - DNA, 1722 bp, Artificial
  insect cell codon optimized RSV F
atggaactccctatccttaagacaaacgcaatcacaactatcctggcggcagtgactttgtgcttcgcctcttctcaaaacattactgaggagttctaccaatccacttgctccgctgtgtcgaaaggctacttgtctgcccttaggaccggctggtacacttccgtgatcactatcgaactctctaacatcaaggagaacaaatgcaacggaacggacgcgaaggtcaagttgatcaagcaggagctggacaagtacaagaacgccgtgaccgaactgcagcttctcatgcaatcgacccctgctgccaataaccgtgctcgtcgtgagctcccacgtttcatgaattacaccctgaacaacactaagaacaacaacgtgaccctgagtaaaaagcgtaaaagacgttttcttggctttctgctcggagtaggaagcgctattgcttctggtattgctgtaagtaaagtgctgcatctcgagggtgaagtgaacaaaatcaaaagtgccctgttgtcgaccaacaaggccgtagtttctctgtctaacggtgtcagtgtcctgacatccaaagtccttgatctgaagaattacatcgacaaacagctgctccccatcgtaaacaagcaatcctgctccataagcaatatcgaaaccgtcatcgagttccagcagaagaacaataggttgcttgaaatcacaagggaattttcagtcaacgccggagtcaccacacccgtgtccacatacatgcttactaactccgagttgctttctctgatcaacgacatgcctatcacaaacgaccagaagaagctgatgagcaacaacgttcagattgtccgtcaacagtcttactcgattatgtctattatcaaggaggaagtattggcatacgttgttcagctcccgctttacggtgtcatcgacactccgtgctggaagctgcatacgtcccctctgtgcacgacaaacacaaaagaaggaagtaacatctgcctcacaaggaccgatcgtggatggtactgtgacaatgccggatcagtctcgttctttccccaggctgagacttgtaaggtgcagtcgaatcgtgttttctgtgacaccatgaactctctgaccttgccctcagaggtaaatttgtgtaacatcgacattttcaaccctaaatacgactgcaagattatgacttccaaggccgatgtgagctcgtccgtaatcacaagtctcggagcgatcgtcagttgctacggcaagactaagtgcaccgctagcaataagaatagaggcattataaagactttctctaacggttgtgactacgtgtcaaacaaaggcgtagacaccgtgtccgtcggtaacacactgtactacgtgaacaagcaagagggtaaatctctctacgttaaaggcgagccgatcattaatttctatgatcccttggtattcccttccgatgagttcgacgcttctatctcgcaagtcaatgagaagatcaaccagtccttggccttcatcagaaaaagcgacgagctcctccacaacgtgaacgttggcaaatcaactaccaacattatgatcaccacgattatcatagtcatcatcgttatcctgctgctcctgatcgctgtgggcctcttcctgtactgcaaggcccgctctacccctgtgaccctttctaaggatcagctgtcgggtattaacaacattgccttttcaaat

SEQ ID NO: 38 - DNA, 1722 bp, Artificial
  mammalian cell codon optimized RSV F
atggagttgcccatcctgaagaccaatgcaattaccaccatcctcgccgccgtgactctgtgtttcgcatcaagtcagaacataaccgaggagttctatcaatcaacctgttctgccgtctccaagggctacctgtctgcactgcgaactgggtggtacacaagcgtcattacaatcgaactgagcaatattaaggaaaataagtgcaatggcaccgacgccaaggtgaagctgatcaaacaggagctggacaaatacaagaacgctgtcaccgaattgcaactgcttatgcagagcacccccgctgccaataacagagcaaggcgcgagctgccgagattcatgaactacaccctgaataacacgaagaataataacgtaaccctgagcaagaagaggaaacgcaggttcctgggttttctgctcggggtgggctcagccatcgcctccggcatcgcagtttctaaggtcctgcatctggagggcgaagtgaataagataaaatcagccctgctgtccacaaacaaggcggttgtgagcctgagtaacggcgtaagcgttctcacctccaaggtgctcgacctgaagaactatattgacaaacagctgctgccaatcgtcaacaaacagtcatgctcaattagcaatattgagactgtgattgaatttcagcagaaaaacaacaggctcctcgagatcactcgggagttcagcgtgaatgcgggggtgacgacccctgtttcaacttacatgctgaccaattctgaattgctctccctcatcaacgacatgccaatcaccaatgaccagaaaaagctgatgagtaacaacgttcagattgttcgccaacagagctatagcatcatgagtatcattaaggaggaggtcctggcctatgttgtacagctcccgctctatggcgtgattgatactccgtgctggaagctgcacacctcaccactctgtactactaacactaaagagggaagcaacatttgtctgacccggaccgaccggggatggtattgcgacaacgctgggagcgtgtctttcttccctcaagcagaaacttgcaaggtacagagtaaccgggtgttttgtgacactatgaatagccttacactgcccagtgaggttaatctgtgtaacatcgacatcttcaatcctaaatacgactgcaaaattatgacgagcaaggctgatgtgtcatcttcagtcataaccagtctgggtgctatcgtttcctgctatgggaagaccaaatgcaccgcatcaaacaaaaaccggggcattatcaaaacattttctaacggatgcgattacgtgtctaataagggggtcgacactgtatctgtggggaacaccctttactatgtgaacaagcaagagggaaaatctctgtacgtgaaaggtgagcctatcataaatttctatgatccgctggtctttccctctgatgaatttgatgcttccattagtcaggtgaatgagaagattaaccaatcactcgcctttatccggaagtcagatgaactcttgcacaacgtaaatgttgggaaaagcaccaccaacatcatgattactactattatcattgtcattatcgttattctcctcctgctgatagccgtggggttgttcctctactgcaaggccaggtccactcctgtgaccctcagcaaggaccagctgagtggcatcaacaacatcgctttcagtaac

SEQ ID NO: 39 - DNA, 32 bp, Artificial
  primer
gcggatccccaagagctcgagtcaatacatag

SEQ ID NO: 40 - DNA, 28 bp, Artificial
  primer
ggggatccgactgcagcaaggattgtgg

SEQ ID NO: 41 - DNA, 8 bp, Artificial
  primer
gcggatcc

SEQ ID NO: 42 - protein, 8 aa, Artificial
  RSV G stem sequence motif
XXFXXFXF

SEQ ID NO: 43 - protein, 497 aa, Artificial
  human parainfluenza virus 5
LDPAALMQIGVIPTNVRQLMYYTEASSAFIVVKLMPTIDSPISGCNITSISSYNATVTKLLQPIGENLETIRNQLIPTRRRFAGVVIGLAALGVATAAQVTAAVALVKANENAAAILNLKNAIQKTNAAVADVVQATQSLGTAVQAVQDHINSVVSPAITAANCKAQDAIIGSILNLYLTELTTIFHNQITNPALSPITIQALRILLGSTLPTVVEKSFNTQISAAELLSSGLLTGQIVGLDLTYMQMVIKIELPTLTVQPATQIIDLATISAFINNQEVMAQLPTRVMVTGSLIQAYPASQCTITPNTVYCRYNDAQVLSDDTMACLQGNLTRCTFSPVVGSFLTRFVLFDGIVYANCRSMLCKCMQPAAVILQPSSSPVTVIDMYKCVSLQLDNLRFTITQLANVTYNSTIKLESSQILSIDPLDISQNLAAVNKSLSDALQHLAQSDTYLSAIEDKIEEILSKIYHIENEIARIKKLIGEAPGGIEGRHHHHHH

SEQ ID NO: 44 - protein, 40 aa, Artificial
  RSV G polypeptide (A2 strain)
RQNKPPSKPNNDFHFEVFNFVPCSICSNNPTCWAICKRIP

SEQ ID NO: 45 - protein, 40 aa, Artificial
  RSV G polypeptide (B1 strain)
RLKNPPKKPKDDYHFEVFNFVPCSICGNNQLCKSICKTIP

SEQ ID NO: 46 - protein, 40 aa, Artificial
  RSV G polypeptide consensus sequence
RXXXPPXKPXXDXHFEVFNFVPCSICXNNXXCXXICKXIP

(19) United States
(12) Patent Application Publication
(10) Pub. No.: US 2011/0318376 A1
(43) Pub. Date: Dec. 29, 2011

(54) RECOMBINANT EXPRESSION OF SELF-FOLDING NEUTRALIZING EPITOPE-BEARING SUBDOMAINS OF THE RESPIRATORY SYNCYTIAL VIRUS ATTACHMENT AND FUSION PROTEINS

(75) Inventors: Yoshihiko Murata, Pittsford, NY (US); Edward E. Walsh, Pittsford, NY (US)

(73) Assignee: UNIVERSITY OF ROCHESTER, Rochester, NY (US)

(21) Appl. No.: 13/142,184

(22) PCT Filed: Dec. 23, 2009
(86) PCT No.: PCT/US09/69364

§ 371 Date: Sep. 12, 2011

(51) Int. Cl. A61K 39/155; C12N 15/45; C12N 15/63; C12N 1/21; A61P 37/02; C12N 1/19; C12N 5/10; C12N 1/13; C07K 14/135; A61P 31/14; C12N 15/62; C12N 1/15

(52) U.S. Cl. 424/186.1; 536/23.4; 536/23.72; 435/320.1; 435/252.3; 435/252.34; 435/252.33; 435/254.11; 435/254.2; 435/325; 435/348; 435/419; 435/257.2; 435/358; 435/367; 435/352; 435/365; 530/322; 530/324; 530/326

(57) ABSTRACT

The present invention is directed to self-folding, soluble, stable RSV G and F polypeptides that contain a neutralizing epitope. Fusion proteins and immunogenic conjugates containing the RSV G and F polypeptides, along with recombinant transgenes and vectors, and host cells suitable for expression of such genetic constructs are also disclosed. Use of the RSV G and F polypeptides, fusion proteins, immunogenic conjugates, or a pharmaceutical composition containing the same, is contemplated for inducing a protective immune response against RSV.

This application claims the benefit of U.S. Provisional Patent Application Ser. No. 61/140,709, filed Dec. 24, 2008, which is hereby incorporated by reference in its entirety.

This invention was made with government support under grant number NIH1R21-AI076781-01A1 awarded by National Institute of Health/National Institute of Allergy and Infection Disease. The government has certain rights in this invention.

### FIELD OF THE INVENTION

The present invention is directed to a Respiratory Syncytial Virus (“RSV”) vaccine based on self-folding, neutralizing epitope bearing subdomains of the RSV attachment (“G”) and fusion (“F”) proteins.

### BACKGROUND OF THE INVENTION

Respiratory syncytial virus is an enveloped virus of the Paramyxoviridae family and its genome is comprised of a non-segmented, (−) single-stranded RNA encoding eleven proteins. The viral envelope bears three transmembrane glycoproteins including the G and F proteins. The G protein plays a key role in viral attachment to the target cell while the F protein is involved in membrane fusion and viral penetration into the host cell. Among viral isolates, some RSV-encoded proteins such as F are highly conserved with respect to amino acid sequence while others such as G display extensive antigenic variation.

Extensive analyses of RSV-induced humoral immune response have shown that only antibodies against F or G proteins are neutralizing and confer resistance to RSV upon passive transfer in animal models. The protective efficacy of several anti-F monoclonal antibodies and their cognate putative binding sites within F has been well characterized in ex vivo and animal models. In contrast, less is known about antibody-antigen binding studies involving G-specific monoclonal antibodies. Anti-G monoclonal antibodies typically exhibit modest neutralizing activity when used individually, but concomitant use of a panel of such monoclonal antibodies can show relatively significant neutralizing activity. This observation is likely due to the genetic variability and extensive post-translational modifications of the RSV G protein.

RSV remains a predictable cause of wintertime respiratory disease among patients of all ages, especially infants and children, the elderly, and the immunocompromised. Despite the burden of RSV-associated disease, therapeutic options are limited and a prophylactic vaccine remains an unmet medical need.

The present invention is directed to overcoming these and other deficiencies in the art.

### SUMMARY OF THE INVENTION

A first aspect of the present invention is directed to an isolated polypeptide comprising a polypeptide fragment of a respiratory syncytial virus (RSV) attachment glycoprotein (G) protein or fusion (F) protein. The polypeptide fragment of the present invention is a self-folding, soluble, and stable fragment that lacks N- or O-glycosylation sites and contains a neutralizing epitope.

A second aspect of the present invention is directed to a fusion protein comprising the polypeptide fragment of the first aspect of the present invention linked by an in-frame fusion to an adjuvant polypeptide.

A third aspect of the present invention is directed to an immunogenic conjugate comprising the polypeptide fragment of the first aspect of the present invention conjugated to an immunogenic carrier molecule.

A fourth aspect of the present invention is directed to an isolated polynucleotide which encodes the polypeptide fragment of the first aspect of the invention or the fusion protein of the second aspect of the present invention.

A fifth aspect of the present invention is directed to a recombinant transgene that comprises the isolated polynucleotide sequence of the fourth aspect of the present invention operably coupled to a promoter-effective DNA molecule, a leader DNA sequence comprising a start-codon, and a transcription termination sequence. Also encompassed by this aspect of the invention are recombinant vectors and host cells containing the transgene or a polynucleotide according to the fourth aspect of the present invention.

A sixth aspect of the present invention is directed to a pharmaceutical composition. This pharmaceutical composition contains the isolated polypeptide, the fusion protein, the immunogenic conjugate, the isolated polynucleotide, or the recombinant transgene of the present invention, along with a pharmaceutically acceptable carrier.

A seventh aspect of the present invention is directed to a method of inducing a neutralizing immune response against respiratory syncytial virus (RSV) in a subject. This method involves administering to the subject a pharmaceutical composition of the present invention in an amount effective to induce a neutralizing immune response against RSV.

RSV causes significant disease burden among pediatric and adult populations, yet an effective vaccine against RSV has not been developed. By generating portions of RSV G and F proteins that self-fold into neutralizing epitope-bearing subdomains and presenting them in the context of a potent adjuvant, the resulting recombinant proteins have the potential to generate a neutralizing humoral immune response, thereby functioning as an effective RSV vaccine. The Examples presented herein demonstrate that the polypeptide fragments of the present invention are reactive with neutralizing antibodies present in sera of RSV-infected patients.

### BRIEF DESCRIPTION OF THE DRAWINGS

FIGS. 1A-1C depict the central unglycosylated region of the RSV G protein. FIG. 1A is a schematic diagram of the G stem (amino acid residues 151-172) and the G loop (amino acid residues 173-190) subdomains. FIG. 1B shows the amino acids comprising the invariant region (amino acid residues 164-176; dotted underline) and the predicted cysteine disulfide bonds between C173-C186 and C176-C182. An alignment of residues comprising the central unglycosylated region (amino acid residues 151-190) of the RGH subtype A5 RSV strain (SEQ ID NO:3) and those from the prototypical subtype A (A2) (SEQ ID NO:44) and B (B1) (SEQ ID NO:45) strains is shown in FIG. 1C. Note that the amino acid residues 164-186, including the invariant amino acid sequence of residues 164-176, are shown in bold. An amino acid consensus sequence of the central unglycosylated region of the G protein based on the RGH, A2, and B1 sequences is also shown in FIG. 1C as SEQ ID NO:46. Residue positions are indicated by numbers above the aligned amino acids.

FIG. 2 is an immunoblot showing GST-RSV G fusion proteins of the present invention. Approximately 3 μg (first two sample lanes) or 0.5-1 μg (remaining sample lanes) of purified GST-G fusion proteins were resolved on 12%/6% SDS-PAGE under reducing conditions and stained with Coomassie Blue R250. For a subset of molecular weight (MW) markers, the corresponding masses (kilodaltons; kD) are indicated to the left. Polypeptide fragments or proteins resolved on each lane are shown on top of the gel. For RSV G-derived moieties, the first and terminal amino acid residue positions derived from the RGH RSV strain are listed; where relevant, G residues from other RSV strains (A2 or B1) are listed in parenthesis. To comparatively demonstrate the purity of the various bacterially derived proteins, two SDS-PAGE gels are shown; the left most three lanes are derived from one gel while the remaining lanes are imaged from another gel.

FIGS. 3A-3D demonstrate that the cognate epitopes of L9 and K6 monoclonal antibodies (mAbs) are localized within the stem region of the RSV G protein. FIGS. 3A-3C are representative immunoblots in which purified RSV G protein (G) (subtype A), GST alone (GST), or GST-RSV G fusion proteins (˜0.5 μm/protein; G-derived residues listed above each corresponding lane) were resolved on denaturing 12%/6% SDS-PAGE gels, transferred onto nitrocellulose, and probed with L9 or K6 mAbs at 1:5,000 dilution followed by goat α-mouse HRP conjugated antibodies (1:20,000 dilution) and chemiluminescence. For each gel, molecular weight (MW) markers are resolved on the left most lane and the corresponding MW masses (in kilodaltons (kD)) are indicated on the left. Note that in FIG. 3A, two independent preparations of GST-G173-190, one from DH5α (D) and another from Origami 2 (O) bacterial strains, were tested for reactogenicity, and that a doublet in GST-G151-190 lanes was occasionally observed. Note also that in FIG. 3C, amino acid residues 151-172 derived from the RGH, A2, or B1 RSV strains (as shown in lane assignments) were tested for recognition by L9 and K6 mAbs. FIG. 3D shows that the L9 and K6 mAb epitopes are overlapping within RSV G 151-172. To the right are the various schematic diagrams, each representing a portion of the RSV G unglycosylated region that was expressed as fusion proteins in bacteria. To the left is a table summarizing the immunoblot data using both mAbs. (+) indicates reproducible mAb recognition of the RSV G derived residues while (−) indicates the absence of detectable mAb binding.

FIG. 4 is a graph depicting the interactions of various GST-RSV G fusion proteins with K6 mAbs as defined by ELISA analysis. Purified G protein from RSV A2-infected HEp2 cells or various bacterially synthesized proteins (GST, GST-G162-172, or GST-G151-172 from RGH, A2, or B1 strains) were coated onto 96 well plates and tested for reactogenicity against K6 mAb. Horizontal axis represents serial two-fold dilutions of K6 mAb starting at 1:5,000 and vertical axis represents OD405 nm of the resulting reactions.

FIGS. 5A-5B are nucleotide sequences encoding the RGH RSV G amino acid sequence of SEQ ID NO:1. FIG. 5A shows a non-codon optimized nucleotide sequence (SEQ ID NO:34) and FIG. 5B shows an insect cell codon optimized nucleotide sequence (SEQ ID NO:35).

FIGS. 6A-6F are nucleotide sequences encoding the RSV G polypeptide fragments of the present invention. FIGS. 6A-B show non-codon optimized (SEQ ID NO:10) and insect cell codon optimized (SEQ ID NO:11) nucleotide sequences, respectfully, encoding the RGH RSV G polypeptide of SEQ ID NO:3 (corresponding to amino acids 151-190 of SEQ ID NO:1). FIGS. 6C-D show non-codon optimized (SEQ ID NO:12) and insect cell codon optimized (SEQ ID NO:13) nucleotide sequences, respectfully, encoding the RGH RSV G polypeptide of SEQ ID NO:4 (corresponding to amino acids 151-186 of SEQ ID NO:1). FIGS. 6E-F show non-codon optimized (SEQ ID NO:14) and insect cell codon optimized (SEQ ID NO:15) nucleotide sequences, respectfully, encoding the RGH RSV G polypeptide of SEQ ID NO:5 (corresponding to amino acids 151-172 of SEQ ID NO:1).

FIG. 7 is a non-codon optimized nucleotide sequence (SEQ ID NO:36) encoding the RGH RSV F amino acid sequence of SEQ ID NO:2.

FIG. 8 is an insect cell codon optimized nucleotide sequence (SEQ ID NO:37) encoding the RGH RSV F amino acid sequence of SEQ ID NO:2.

FIG. 9 is a mammalian cell codon optimized nucleotide sequence (SEQ ID NO:38) encoding the RGH RSV F amino acid sequence of SEQ ID NO:2.

FIGS. 10A-10C are nucleotide sequences encoding the RSV F polypeptide fragment of the present invention corresponding to amino acids 382-459 of the RGH RSV F protein (SEQ ID NO:7). These nucleotide sequences include a non-codon optimized nucleotide sequence (SEQ ID NO:16; FIG. 10A), an insect cell codon optimized nucleotide sequence (SEQ ID NO:17; FIG. 10B), and a mammalian cell codon optimized nucleotide sequence (SEQ ID NO:18; FIG. 10C).

FIGS. 11A-11C are nucleotide sequences encoding the RSV F polypeptide fragment of the present invention corresponding to amino acids 382-459 of the RGH RSV F protein (SEQ ID NO:7) having a cysteine to serine substitution at amino acid position 439 (amino acid position 58 of SEQ ID NO:7). These nucleotide sequences include a non-codon optimized nucleotide sequence (SEQ ID NO:19; FIG. 11A), an insect cell codon optimized nucleotide sequence (SEQ ID NO:20; FIG. 11B), and a mammalian cell codon optimized nucleotide sequence (SEQ ID NO:21; FIG. 11C). The cysteine to serine substitution is shown in bold and underlined.

FIGS. 12A-12C are nucleotide sequences encoding the RSV F polypeptide fragment of the present invention corresponding to amino acids 233-303 of the RGH RSV F protein (SEQ ID NO:8). These nucleotide sequences include a non-codon optimized nucleotide sequence (SEQ ID NO:22; FIG. 12A), an insect cell codon optimized nucleotide sequence (SEQ ID NO:23; FIG. 12B), and a mammalian cell codon optimized nucleotide sequence (SEQ ID NO:24; FIG. 12C).

FIGS. 13A-13C are nucleotide sequences encoding the RSV F polypeptide fragment of the present invention corresponding to amino acids 233-285 of the RGH RSV F protein (SEQ ID NO:9). These nucleotide sequences include a non-codon optimized nucleotide sequence (SEQ ID NO:25; FIG. 13A), an insect cell codon optimized nucleotide sequence (SEQ ID NO:26; FIG. 13B), and a mammalian cell codon optimized nucleotide sequence (SEQ ID NO:27; FIG. 13C).

FIGS. 14A-14C are nucleotide sequences encoding the RSV F polypeptide fragment of the present invention corresponding to amino acids 374-459 of the RGH RSV F protein (SEQ ID NO:6). These nucleotide sequences include a non-codon optimized nucleotide sequence (SEQ ID NO:28; FIG. 14A), an insect cell codon optimized nucleotide sequence (SEQ ID NO:29; FIG. 14B), and a mammalian cell codon optimized nucleotide sequence (SEQ ID NO:30; FIG. 14C).

FIGS. 15A-15C are nucleotide sequences encoding the RSV F polypeptide fragment of the present invention corresponding to amino acids 374-459 of the RGH RSV F protein (SEQ ID NO:6) having a cysteine to serine substitution at amino acid position 439 (amino acid position 66 in SEQ ID NO:6). These nucleotide sequences include a non-codon optimized nucleotide sequence (SEQ ID NO:31; FIG. 15A), an insect cell codon optimized nucleotide sequence (SEQ ID NO:32; FIG. 15B), and a mammalian cell codon optimized nucleotide sequence (SEQ ID NO:33; FIG. 15C). The cysteine to serine substitution is shown in bold and underlined.

FIG. 16 shows a sequence alignment between the amino acid sequence of the RGH strain of the RSV F protein (“query”) (SEQ ID NO:2) and the amino acid sequence of the prefusion conformation of the human parainfluenza virus 5 (PIV5) (Protein Data Base (PDB) structure database accession reference code 2B9B_A; SEQ ID NO:43). This alignment represents the most significant match between the RSV F amino acid sequence and an amino acid sequence encoding a protein with a known structure. This alignment was used for deriving the RSV F structural predictions disclosed herein.

FIGS. 17A-17B are ribbon diagrams of the structural domain of the PIV5 F protein illustrating regions of homology with the RSV F antigenic domains IV/V/VI. The ribbon diagram of FIG. 17A shows the RSV antigenic domains IV/V/VI (including amino acid residues 423-436) mapping to amino acid residues 365-378 within a surface-exposed region of structural domain II of PIV5 F protein. FIG. 17B shows the PIV5 structural domain II alone.

FIG. 18 is ribbon diagram of the RSV F protein (SEQ ID NO:2) modeling the predicted formation of disulfide bonds between the cysteine residues (shown as grey balls) at amino acid positions 382-393, positions 416-422 and positions 37-439 (Day et al., “Contribution of Cysteine Residues in the Extracellular Domain of the F Protein of Human Respiratory Syncytial Virus to its Function,” Virol J 3:34 (2006), which is hereby incorporated by reference in its entirety). Other cysteine residues are also depicted, with specific residue disulfide pairs indicated on the monomer.

FIGS. 19A-19B are ribbon diagrams illustrating homology between RSV F antigenic domain II and the corresponding structural region of PIV5 F protein. The ribbon diagram of FIG. 19A shows the RSV F antigenic domain II (including amino acids 255-278) mapping to amino acids 203-227 within the entire PIV5 monomer. FIG. 19B shows the homologous regions alone.

FIG. 20 is a ribbon diagram showing the RSV F antigenic domain I (amino acid 389; arrow) mapping to a surface exposed region between structural helix 2 and structural domain II of the PIV 5 protein.

### DETAILED DESCRIPTION OF THE INVENTION

A first aspect of the present invention is directed to an isolated polypeptide consisting of a polypeptide fragment of a respiratory syncytial virus (RSV) attachment glycoprotein (G) protein or fusion (F) protein. The polypeptide fragment of the present invention is a self-folding, soluble, and stable fragment that lacks N- or O-glycosylation sites and comprises a neutralizing epitope.

As used herein, a “self-folding” polypeptide refers to a polypeptide that folds into a discrete, independent structural domain of the RSV G or RSV F proteins when in solution. The self-folded polypeptides of the present invention are also soluble and stable in aqueous or mixed aqueous/organic solutions which are at or near physiological pH (7.0-7.4) and/or ionic strength (similar to 1× phosphate buffered saline, pH. 7.4, 8.06 mM sodium phosphate, 1.94 mM potassium phosphate, 2.7 mM potassium chloride, and 137 mM sodium chloride).

As used herein, a “neutralizing epitope” encompasses an epitope that induces an antibody response that is capable of neutralizing the activity of the RSV F or G proteins, respectively. The neutralizing activity of an antibody generated in response to a neutralizing epitope can be assessed using an in vitro assay or an in vivo assay (e.g., RSV animal challenge with passive transfer of relevant antibodies).

In accordance with this aspect of the present invention, the isolated polypeptide of the present invention is preferably less than 90 amino acids in length. More preferably, the isolated polypeptide is less than 85 amino acids in length, and most preferably, the isolated polypeptide is less than 80 amino acids in length. The isolated polypeptide of the present invention is preferably greater than 20 amino acids in length.

Because of its inherent genetic variability, the full-length of the RSV G protein varies among clinical isolates; the typical length being 289-299 amino acids, although recent isolates may be as long as 310 amino acids (“aa”). The majority of the genetic variability of the G protein occurs near its carboxy-terminus (Melero et al., “Antigenic Structure, Evolution, and Immunobiology of Human Respiratory Syncytial Virus Attachment (G) Protein,” J Gen Virol 78:2411-2418 (1997), which is hereby incorporated by reference in its entirety). The G protein is extensively glycosylated and bears a hydrophobic domain (aa 38-66) that is thought to function as a transmembrane (“TM”) domain near its amino-terminus. The G protein is thought to exist in one of two forms: a membrane-bound homotetramer in which monomers are likely juxtaposed to one another near their respective TM domains; and a shorter, secreted form of G that is devoid of the TM domain by virtue of an alternate initiation methionine at amino acid position 48 and subsequent proteolytic cleavage after amino acid position 65. The latter form of G is thought to function as an immunomodulatory factor within the host during RSV infection.

The hallmarks of all G protein amino acid sequences isolated to date include: (i) the existence of a central region of amino acids (typically aa 150-197; the boundaries are approximate and strain-dependent given the genetic variability of G) that bears no glycosylation signals; (ii) aa 164-176 that appears to be invariantly conserved among all RSV isolates; and (iii) the presence of four cysteines (aa 173, 176, 182, and 186) which bear similarities to the CX3C motif and likely form two disulfide bonds in a loop configuration as demonstrated by NMR studies. Several reports in the literature note that aside from this central G loop region, there appear to be no other significant structural motifs, and no crystal structure of G has been reported (Melero et al., “Antigenic Structure, Evolution, and Immunobiology of Human Respiratory Syncytial Virus Attachment (G) Protein,” J Gen Virol 78:2411-2418 (1997); Martinez et al., “Antigenic Structure of the Human Respiratory Syncytial Virus G Glycoprotein and Relevance of Hypermutation Events for the Generation of Antigenic Variants,” J Gen Virol 78:2419-2429 (1997); and Langedijk et al., “Proposed Three-dimensional Model for the Attachment Protein G of Respiratory Syncytial Virus,” J Gen Virol 77:1249-1257 (1996), which are hereby incorporated by reference in their entirety).

Based primarily on the G amino acid sequences of RSV strains selected to grow in the presence of neutralizing antibodies, there appears to exist a number of potential amino acid sequences that are involved in interactions with such antibodies. However, because of the genetic variability that is inherent to G protein, most neutralizing antibodies likely recognize virus strain-specific epitopes with limited ability to cross-recognize a panel of clinical isolates. A central region of G (aa 130-230 from the RSV Long strain) has been expressed in E. coli, and when conjugated to the albumin-binding domain of streptococcal protein G (termed BBG2Na), was shown to elicit a protective response against RSV intranasal challenge in mice (Plotnickey-Gilquin et al., “Identification of Multiple Protective Epitopes (protectopes) in the Central Conserved Domain of a Prototype Human Respiratory Syncytial Virus G Protein,” J Virol 73:5637-5645 (1999), which is hereby incorporated by reference in its entirety). Analysis of monoclonal antibodies generated from these mice revealed short, putative protective epitopes (aa 152-163, 165-172, 171-187, and 194-206) within aa 130-230 of G protein. Except for peptide 171-187 bearing four cysteines that formed the expected loop structure with two disulfide bonds, none of the other four B cell epitopes were shown to have any significant structure in solution based on CD and NMR data.

According to one embodiment, the polypeptide of the present invention is derived from the amino acid sequence of the RGH strain of the RSV G protein (genotype A5, isolated in 1999) having an amino acid sequence of SEQ ID NO:1 shown below.

```
Met Ser Lys Thr Lys Asp Gln Arg Thr Ala Lys Thr Leu Glu Lys Thr
1 5 10 15
Trp Asp Thr Leu Asn His Leu Leu Phe Ile Ser Ser Cys Leu Tyr Lys
                        20 25 30
Leu Asn Leu Lys Ser Ile Ala Gln Ile Thr Leu Ser Ile Leu Ala Met
                35 40 45
Ile Ile Ser Thr Ser Leu Ile Ile Val Ala Ile Ile Phe Ile Ala Ser
        50 55 60
Ala Asn Asn Lys Val Thr Leu Thr Thr Ala Ile Ile Gln Asp Ala Thr
65 70 75 80
Ser Gln Ile Lys Asn Thr Thr Pro Thr Tyr Leu Thr Gln Asn Pro Gln
                                85 90 95
Leu Gly Ile Ser Phe Phe Asn Leu Ser Gly Thr Ile Ser Gln Thr Thr
                        100 105 110
Ala Ile Leu Ala Leu Thr Thr Pro Ser Val Glu Ser Ile Leu Gln Ser
                115 120 125
Thr Thr Val Lys Thr Lys Asn Thr Thr Thr Thr Gln Ile Gln Pro Ser
        130 135 140
Lys Pro Thr Thr Lys Gln Arg Gln Asn Lys Pro Pro Asn Lys Pro Asn
145 150 155 160
Asp Asp Phe His Phe Glu Val Phe Asn Phe Val Pro Cys Ser Ile Cys
                                165 170 175
Ser Asn Asn Pro Thr Cys Trp Ala Ile Cys Lys Arg Ile Pro Ser Lys
                        180 185 190
Lys Pro Gly Lys Lys Thr Thr Thr Lys Pro Thr Lys Lys Pro Thr Ile
                195 200 205
Lys Thr Thr Lys Lys Asp Leu Lys Pro Gln Thr Thr Lys Pro Lys Glu
        210 215 220
Ala Pro Thr Thr Lys Pro Thr Glu Lys Pro Thr Ile Asn Ile Thr Lys
225 230 235 240
Pro Asn Ile Arg Thr Thr Leu Leu Thr Asn Ser Thr Thr Gly Asn Leu
                                245 250 255
Glu His Thr Ser Gln Glu Glu Thr Leu His Ser Thr Ser Ser Glu Ser
                        260 265 270
Ser Thr Ser Pro Ser Gln Ile Tyr Thr Thr Ser Glu Tyr Leu Ser Gln
                275 280 285
Pro Pro Ser Pro Ser Asn Ile Thr Asp Gln
        290 295
```

In one embodiment, the isolated polypeptide comprises a polypeptide fragment corresponding to amino acids 151-190 of SEQ ID NO:1 (SEQ ID NO:3):

```
Arg Gln Asn Lys Pro Pro Asn Lys Pro Asn Asp Asp Phe His Phe Glu
1 5 10 15
Val Phe Asn Phe Val Pro Cys Ser Ile Cys Ser Asn Asn Pro Thr Cys
                        20 25 30
Trp Ala Ile Cys Lys Arg Ile Pro
                35 40
```

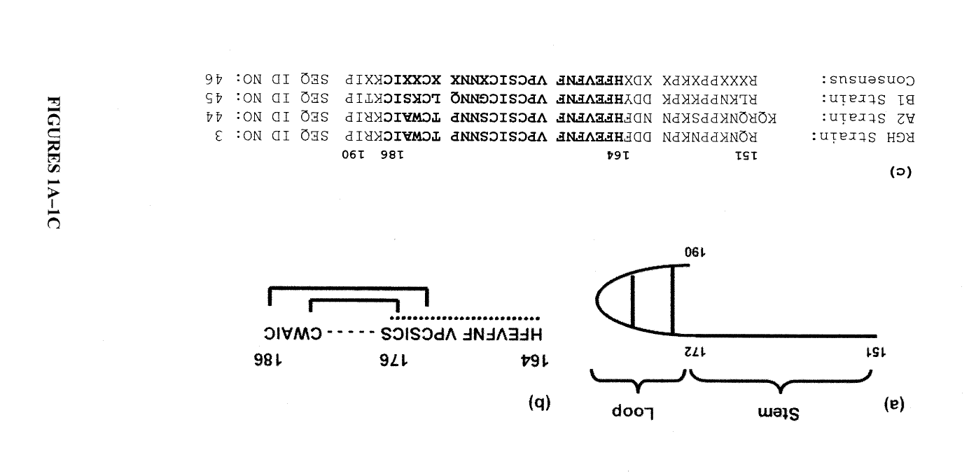

Alternatively, the isolated polypeptide comprising a polypeptide fragment corresponding to amino acids 151-190 of SEQ ID NO:1 has the amino acid sequence of SEQ ID NO:44, SEQ ID NO:45, or SEQ ID NO:46 as shown in FIG. 1C.

In an alternative embodiment, the polypeptide fragment has an amino acid sequence corresponding to amino acids 151-186 of SEQ ID NO:1 (SEQ ID NO:4):

```
Arg Gln Asn Lys Pro Pro Asn Lys Pro Asn Asp Asp Phe His Phe Glu
1 5 10 15
Val Phe Asn Phe Val Pro Cys Ser Ile Cys Ser Asn Asn Pro Thr Cys
                        20 25 30
Trp Ala Ile Cys
                35
```

In another embodiment, the polypeptide fragment of the present invention has an amino acid sequence corresponding to amino acids 151-172 of SEQ ID NO:1 (SEQ ID NO:5):

Arg Gln Asn Lys Pro Pro Asn Lys Pro Asn Asp Asp Phe His Phe Glu
1 5 10 15
Val Phe Asn Phe Val Pro
20

In accordance with this aspect of the invention, the polypeptide fragments of the RSV G protein contain no putative N- or O-glycosylation sites. The absence of glycosylation increases the cross-neutralizing capacity of the antibodies generated against these isolated polypeptide fragments. The above-described polypeptides of the RSV G protein are predicted to be self-folding, soluble, and stable, as defined above.

In one embodiment of the present invention, the isolated polypeptides of the RSV G protein contain at least two cysteine residues, these cysteine residues being located at amino acid positions 173 and 176 of SEQ ID NO:1. Alternatively, the isolated polypeptide fragments of the RSV G protein contain four cysteine residues, these cysteine residues being located at amino acids positions 173, 176, 182, and 186 of SEQ ID NO:1.

In one embodiment of the present invention, isolated polypeptides of the RSV G protein consisting of amino acid residues 1-41 of SEQ ID NO:44 and/or fragments there of, such as fragments consisting of amino acid residues 6-23 and 6-24 of SEQ ID NO:44, are excluded from the present invention.

The nascent RSV Fₒ protein is cleaved by intracellular proteases to generate two subunits, F1 (˜50 kilodaltons (kD)) and F2 (˜20 kD), that are covalently linked by a disulfide bond. Recent studies have extensively characterized other putative disulfide bonds within and between the F1-F2 subunits (Day et al., “Contribution of Cysteine Residues in the Extracellular Domain of the F Protein of Human Respiratory Syncytial Virus to its Function,” Virol J 3:34-45 (2006), which is hereby incorporated by reference in its entirety). The amino-termini of F1 and F2 are predicted to be hydrophobic in nature, the former containing the fusion peptide, a contiguous amino acid sequence that is essential for viral fusion. The F1 subunit contains several structural motifs, including the heptad repeats A and B (HRA and HRB), which are involved in conformational changes of F protein during membrane fusion, the TM domain, and a short cytoplasmic tail domain. As in the case of the F proteins from related viruses, e.g., Newcastle disease virus (NDV) and human parainfluenzavirus 5 (PIV5), the RSV F protein in its native conformation exists as a homomeric trimer in a “metastable,” pre-fusion state. At the time of membrane juxtaposition, the F protein structure undergoes significant conformational changes to a lower energy state (post-fusion state) (Chen et al., “The Structure of the Fusion Glycoprotein of Newcastle Disease Virus Suggests a Novel Paradigm for the Molecular Mechanism of Membrane Fusion,” Structure 9:255-266 (2001); Lin et al., “Cloning, Expression, and Crystallization of the Fusion Protein of Newcastle Disease Virus,” Virology 290:290-299 (2001); Yin et al., “Structure of the Uncleaved Ectodomain of the Paramyxovirus (hPIV3) Fusion Protein,” PNAS 102:9288-9293 (2005); and Yin et al., “Structure of the Parainfluenza Virus 5 F Protein in its Metastable, Prefusion Conformation,” Nature 439:38-44 (2006), which are hereby incorporated by reference in their entirety).

The neutralizing epitopes within the F protein have been immunologically and structurally mapped to three antigenic domains: domain I (involving amino acid 389), domain II (amino acids 262-275, including the putative binding sites of palivizumab, a neutralizing mAb that is licensed for clinical use against RSV in high-risk populations), and domain IV/V/VI (a contiguous series of amino acids which collectively bears the putative binding sites of a number of neutralizing mAbs) (Lopez et al., “Antigenic Structure of Human Respiratory Syncytial Virus Fusion Glycoprotein,” J Virol 72:6922-6928 (1998); Lieu et al., “Relationship Between the Loss of Neutralizing Antibody Binding and Fusion Activity of the F Protein of Human Respiratory Syncytial Virus,” Virol J 4:71-5 (2007); Smith et al., “Modeling the Structure of the Fusion Protein from Human Respiratory Syncytial Virus,” Protein Engineering 15:365-371 (2002); and Wu et al., “Characterization of the Epitope for Anti-human Respiratory Syncytial Virus F Protein Monoclonal Antibody 101F Using Synthetic Peptides and Genetic Approaches,” J Gen Virol 88:2719-2723 (2007), which are hereby incorporated by reference in their entirety). Of the three neutralizing epitope-bearing portions of the RSV F protein, antigenic domain II has been the most extensively characterized based on its helix-loop-helix conformation (amino acids 255-275) in solution (Toiron et al., “Conformational Studies of a Short Linear Peptide Corresponding to a Major Conserved Neutralizing Epitope of Human Respiratory Syncytial Virus Fusion Glycoprotein,” Biopolymers 39:537-548 (1996), which is hereby incorporated by reference in its entirety) and the presence of amino acid mutations within this region that are found among several palivizumab-resistant RSV strains.

According to one embodiment, the polypeptide of the present invention is derived from the amino acid sequence of the RGH strain of the RSV F protein having an amino acid sequence of SEQ ID NO:2 shown below.

```
Met Glu Leu Pro Ile Leu Lys Thr Asn Ala Ile Thr Thr Ile Leu Ala
1 5 10 15
Ala Val Thr Leu Cys Phe Ala Ser Ser Gln Asn Ile Thr Glu Glu Phe
                        20 25 30
Tyr Gln Ser Thr Cys Ser Ala Val Ser Lys Gly Tyr Leu Ser Ala Leu
                35 40 45
Arg Thr Gly Trp Tyr Thr Ser Val Ile Thr Ile Glu Leu Ser Asn Ile
        50 55 60
Lys Glu Asn Lys Cys Asn Gly Thr Asp Ala Lys Val Lys Leu Ile Lys
65 70 75 80
Gln Glu Leu Asp Lys Tyr Lys Asn Ala Val Thr Glu Leu Gln Leu Leu
                                85 90 95
Met Gln Ser Thr Pro Ala Ala Asn Asn Arg Ala Arg Arg Glu Leu Pro
                        100 105 110
Arg Phe Met Asn Tyr Thr Leu Asn Asn Thr Lys Asn Asn Asn Val Thr
                115 120 125
Leu Ser Lys Lys Arg Lys Arg Arg Phe Leu Gly Phe Leu Leu Gly Val
        130 135 140
Gly Ser Ala Ile Ala Ser Gly Ile Ala Val Ser Lys Val Leu His Leu
145 150 155 160
Glu Gly Glu Val Asn Lys Ile Lys Ser Ala Leu Leu Ser Thr Asn Lys
                                165 170 175
Ala Val Val Ser Leu Ser Asn Gly Val Ser Val Leu Thr Ser Lys Val
                        180 185 190
Leu Asp Leu Lys Asn Tyr Ile Asp Lys Gln Leu Leu Pro Ile Val Asn
                195 200 205
Lys Gln Ser Cys Ser Ile Ser Asn Ile Glu Thr Val Ile Glu Phe Gln
        210 215 220
Gln Lys Asn Asn Arg Leu Leu Glu Ile Thr Arg Glu Phe Ser Val Asn
225 230 235 240
Ala Gly Val Thr Thr Pro Val Ser Thr Tyr Met Leu Thr Asn Ser Glu
                                245 250 255
Leu Leu Ser Leu Ile Asn Asp Met Pro Ile Thr Asn Asp Gln Lys Lys
                        260 265 270
Leu Met Ser Asn Asn Val Gln Ile Val Arg Gln Gln Ser Tyr Ser Ile
                275 280 285
Met Ser Ile Ile Lys Glu Glu Val Leu Ala Tyr Val Val Gln Leu Pro
        290 295 300
Leu Tyr Gly Val Ile Asp Thr Pro Cys Trp Lys Leu His Thr Ser Pro
305 310 315 320
Leu Cys Thr Thr Asn Thr Lys Glu Gly Ser Asn Ile Cys Leu Thr Arg
                                325 330 335
Thr Asp Arg Gly Trp Tyr Cys Asp Asn Ala Gly Ser Val Ser Phe Phe
                        340 345 350
Pro Gln Ala Glu Thr Cys Lys Val Gln Ser Asn Arg Val Phe Cys Asp
                355 360 365
Thr Met Asn Ser Leu Thr Leu Pro Ser Glu Val Asn Leu Cys Asn Ile
        370 375 380
Asp Ile Phe Asn Pro Lys Tyr Asp Cys Lys Ile Met Thr Ser Lys Ala
385 390 395 400
Asp Val Ser Ser Ser Val Ile Thr Ser Leu Gly Ala Ile Val Ser Cys
                                405 410 415
Tyr Gly Lys Thr Lys Cys Thr Ala Ser Asn Lys Asn Arg Gly Ile Ile
                        420 425 430
Lys Thr Phe Ser Asn Gly Cys Asp Tyr Val Ser Asn Lys Gly Val Asp
                435 440 445
Thr Val Ser Val Gly Asn Thr Leu Tyr Tyr Val Asn Lys Gln Glu Gly
        450 455 460
Lys Ser Leu Tyr Val Lys Gly Glu Pro Ile Ile Asn Phe Tyr Asp Pro
465 470 475 480
Leu Val Phe Pro Ser Asp Glu Phe Asp Ala Ser Ile Ser Gln Val Asn
                                485 490 495
Glu Lys Ile Asn Gln Ser Leu Ala Phe Ile Arg Lys Ser Asp Glu Leu
                        500 505 510
Leu His Asn Val Asn Val Gly Lys Ser Thr Thr Asn Ile Met Ile Thr
                515 520 525
Thr Ile Ile Ile Val Ile Ile Val Ile Leu Leu Leu Leu Ile Ala Val
        530 535 540
Gly Leu Phe Leu Tyr Cys Lys Ala Arg Ser Thr Pro Val Thr Leu Ser
545 550 555 560
Lys Asp Gln Leu Ser Gly Ile Asn Asn Ile Ala Phe Ser Asn
                                565 570
```

In one embodiment, the isolated polypeptide of the present invention mimics a “structural domain II-like region” and comprises a polypeptide fragment corresponding to amino acids 374-459 of SEQ ID NO:2 (SEQ ID NO:6).

```
Thr Leu Pro Ser Glu Val Asn Leu Cys Asn Ile Asp Ile Phe Asn Pro
1 5 10 15
Lys Tyr Asp Cys Lys Ile Met Thr Ser Lys Ala Asp Val Ser Ser Ser
                        20 25 30
Val Ile Thr Ser Leu Gly Ala Ile Val Ser Cys Tyr Gly Lys Thr Lys
                35 40 45
Cys Thr Ala Ser Asn Lys Asn Arg Gly Ile Ile Lys Thr Phe Ser Asn
        50 55 60
Gly Cys Asp Tyr Val Ser Asn Lys Gly Val Asp Thr Val Ser Val Gly
65 70 75 80
Asn Thr Leu Tyr Tyr Val
                                85
```

The isolated polypeptide fragment can alternatively comprise an amino acid sequence corresponding to amino acids 382-459 of SEQ ID NO:2 (SEQ ID NO:7).

```
Cys Asn Ile Asp Ile Phe Asn Pro Lys Tyr Asp Cys Lys Ile Met Thr
1 5 10 15
Ser Lys Ala Asp Val Ser Ser Ser Val Ile Thr Ser Leu Gly Ala Ile
                        20 25 30
Val Ser Cys Tyr Gly Lys Thr Lys Cys Thr Ala Ser Asn Lys Asn Arg
                35 40 45
Gly Ile Ile Lys Thr Phe Ser Asn Gly Cys Asp Tyr Val Ser Asn Lys
        50 55 60
Gly Val Asp Thr Val Ser Val Gly Asn Thr Leu Tyr Tyr Val
65 70 75
```

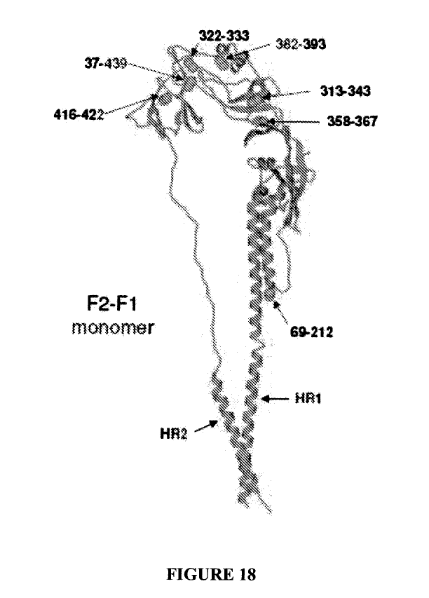

There are five cysteine residues located within amino acids 374-459 of SEQ ID NO:2 (i.e., amino acid residues 382, 393, 416, 422, and 439 of SEQ ID NO:2). FIG. 18 illustrates the predicted disulfide bonds between these cysteine residues within the RSV F protein. The presence of these cysteine residues in the above described RSV F polypeptides facilitate the self-folding capacity of the individual polypeptide fragments. In a preferred embodiment, the cysteine located at amino acid position 66 of SEQ ID NO:6 and the cysteine located at amino acid position 58 of SEQ ID NO:7 are replaced with a serine residue to eliminate the predicted unpaired cysteine residue and increase the self-folding capability of these isolated polypeptide fragments.

In an alternative embodiment of this aspect of the present invention, the polypeptide fragment of the RSV F protein mimics a “structural domain III-like” region and has an amino acid sequence corresponding to amino acids 233-303 of SEQ ID NO:2 (SEQ ID NO:8):

```
Ile Thr Arg Glu Phe Ser Val Asn Ala Gly Val Thr Thr Pro Val Ser
1 5 10 15
Thr Tyr Met Leu Thr Asn Ser Glu Leu Leu Ser Leu Ile Asn Asp Met
                        20 25 30
Pro Ile Thr Asn Asp Gln Lys Lys Leu Met Ser Asn Asn Val Gln Ile
                35 40 45
Val Arg Gln Gln Ser Tyr Ser Ile Met Ser Ile Ile Lys Glu Glu Val
        50 55 60
Leu Ala Tyr Val Val Gln Leu
65 70
```

or amino acids 233-286 of SEQ ID NO:2 (SEQ ID NO:9):

```
Ile Thr Arg Glu Phe Ser Val Asn Ala Gly Val Thr Thr Pro Val Ser
1 5 10 15
Thr Tyr Met Leu Thr Asn Ser Glu Leu Leu Ser Leu Ile Asn Asp Met
                        20 25 30
Pro Ile Thr Asn Asp Gln Lys Lys Leu Met Ser Asn Asn Val Gln Ile
                35 40 45
Val Arg Gln Gln Ser
        50
```

In accordance with this aspect of the invention, the polypeptide fragments of the RSV F protein contain no putative N- or O-glycosylation sites. The absence of glycosylation increases the cross-neutralizing capacity of the antibodies generated against these isolated polypeptide fragments. The above-described polypeptides of the RSV F protein are also self-folding, soluble, and stable in aqueous or mixed aqueous/organic solutions which are at or near physiological pH (7.0-7.4) and/or ionic strength (similar to 1× phosphate buffered saline, pH. 7.4, 8.06 mM sodium phosphate, 1.94 mM potassium phosphate, 2.7 mM potassium chloride, and 137 mM sodium chloride).

In one embodiment of the present invention, the isolated polypeptides of the RSV F protein contain at least two cysteine residues. Alternatively, the isolated polypeptide fragments of the RSV F protein contain at least four cysteine residues.

A second aspect of the present invention relates to a fusion protein including any one of the isolated polypeptide fragments of RSV G or RSV F proteins of the present invention described supra linked by an in-frame fusion to an adjuvant polypeptide.

By way of example, and without limitation, suitable fusion proteins of the present invention include an adjuvant polypeptide fused in-frame to any one of the above listed RSV G polypeptides (e.g., SEQ ID NOS: 3, 4, 5) or RSV F polypeptides (e.g., SEQ ID NOS: 6, 7, 8, 9). The adjuvant polypeptide can be any peptide adjuvant known in art including, but not limited to, flagellin, human papillomavirus (HPV) L1 or L2 proteins, herpes simplex glycoprotein D (gD), complement C4 binding protein, toll-like receptor-4 (TLR4) ligand, and IL-1β.

The RSV G or F fusion proteins of the present invention can be generated using standard techniques known in the art. For example, the fusion polypeptide can be prepared by translation of an in-frame fusion of the polynucleotide sequences encoding the RSV G or F fragments (described infra) and the adjuvant, i.e., a hybrid gene. The hybrid gene encoding the fusion polypeptide is inserted into an expression vector which is used to transform or transfect a host cell. Alternatively, the polynucleotide sequence encoding the RSV G or F polypeptide is inserted into an expression vector in which the polynucleotide encoding the adjuvant is already present. The peptide adjuvant of the fusion protein can be fused to the N-, or preferably, to the C-terminal end of the RSV G or F polypeptide.

Fusions between the RSV G or RSV F polypeptide and the protein adjuvant may be such that the amino acid sequence of the RSV G or RSV F polypeptide is directly contiguous with the amino acid sequence of the adjuvant. Alternatively, the RSV G or RSV F portion may be coupled to the adjuvant by way of a short linker sequence. Suitable linker sequences include glycine rich linkers (e.g., GGGS₂₋₃), serine-rich linkers (e.g., GS_{N}), or other flexible immunoglobulin linkers as disclosed in U.S. Pat. No. 5,516,637 to Huang et al, which is hereby incorporated by reference in its entirety.

Another aspect of the present invention is directed to an immunogenic conjugate including any one of the RSV G or RSV F polypeptide fragments of the present invention conjugated to an immunogenic carrier molecule.

Suitable immunogenic conjugates of the present invention include, but are not limited to, an immunogenic carrier molecule covalently or non-covalently bonded to any one of the above listed RSV G polypeptides (e.g., SEQ ID NOS: 3, 4, 5,) or RSV F polypeptides (e.g., SEQ ID NOS: 6, 7, 8, 9). Any suitable immunogenic carrier molecule can be used. Exemplary immunogenic carrier molecules include, but are in no way limited to, bovine serum albumin, chicken egg ovalbumin, keyhole limpet hemocyanin, tetanus toxoid, diphtheria toxoid, thyroglobulin, a pneumococcal capsular polysaccharide, CRM 197, and a meningococcal outer membrane protein.

Another aspect of the present invention relates to the isolated polynucleotides that encode the above-described isolated RSV G and RSV F polypeptides and the isolated polynucleotides that encode any of the above-described RSV G or RSV F fusion proteins. In a preferred embodiment, the polynucleotide sequences encoding the isolated polypeptides or fusion proteins of the present invention are codon-optimized for expression of the polypeptide in an appropriate host cell, such as a eukaryotic or yeast host cell.

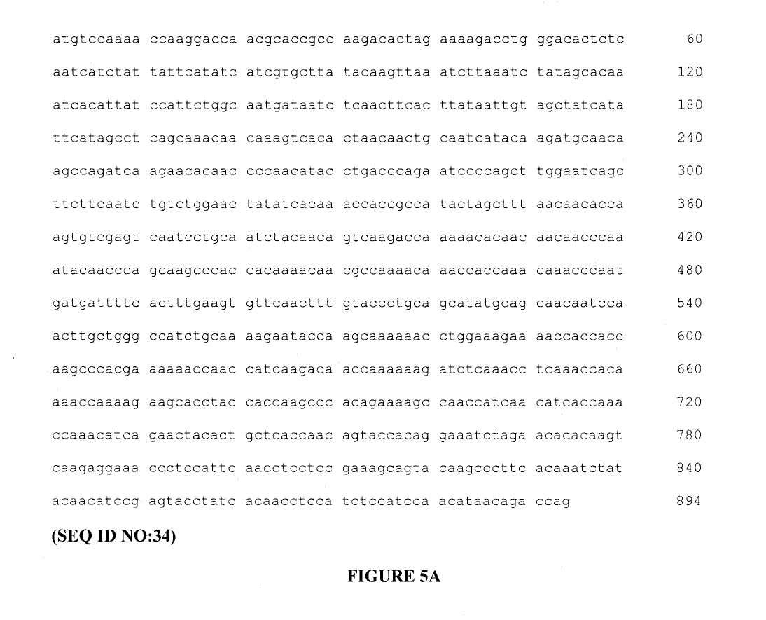

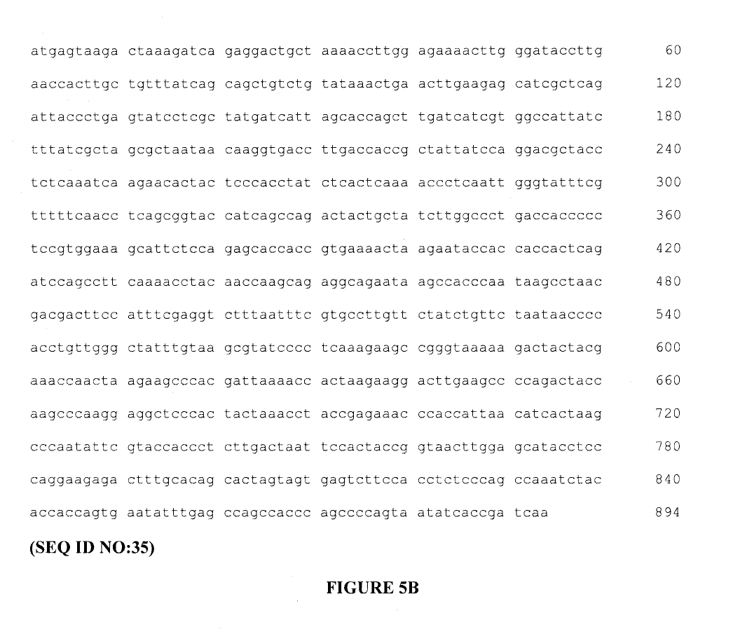

An isolated polynucleotide encoding the amino acid sequence of the RGH strain of RSV G (SEQ ID NO:1) comprises the nucleotide sequence of SEQ ID NO:34 (FIG. 5A). A codon-optimized polynucleotide encoding the RGH RSV G protein of SEQ ID NO:1, suitable for use in insect host cells, comprises the nucleotide sequence of SEQ ID NO:35 (FIG. 5B).

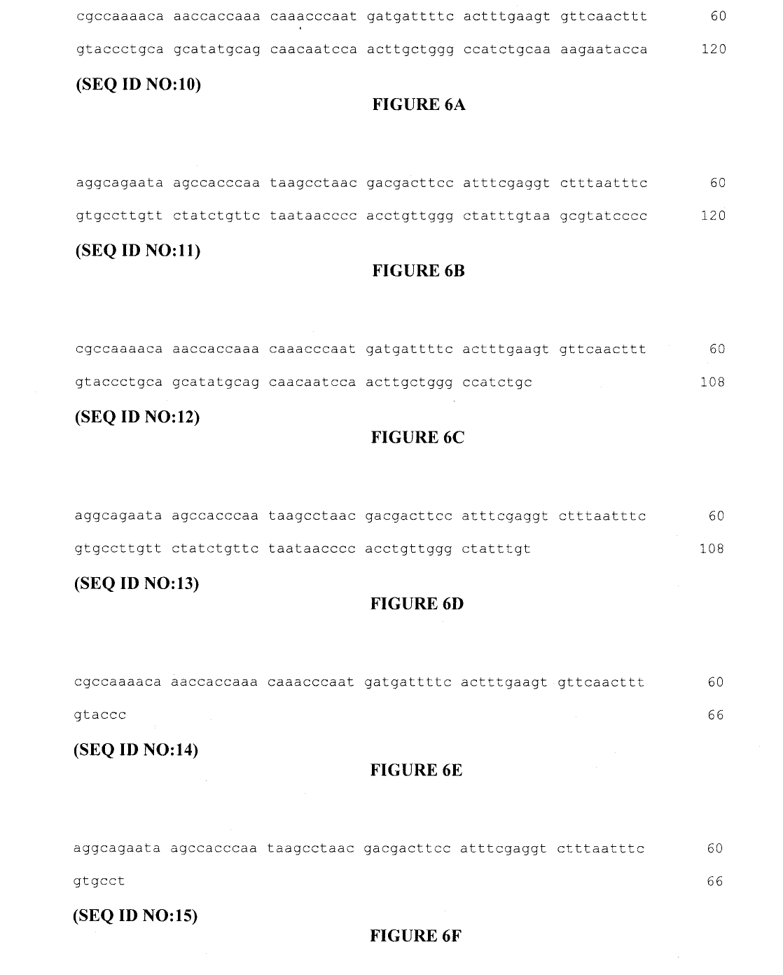

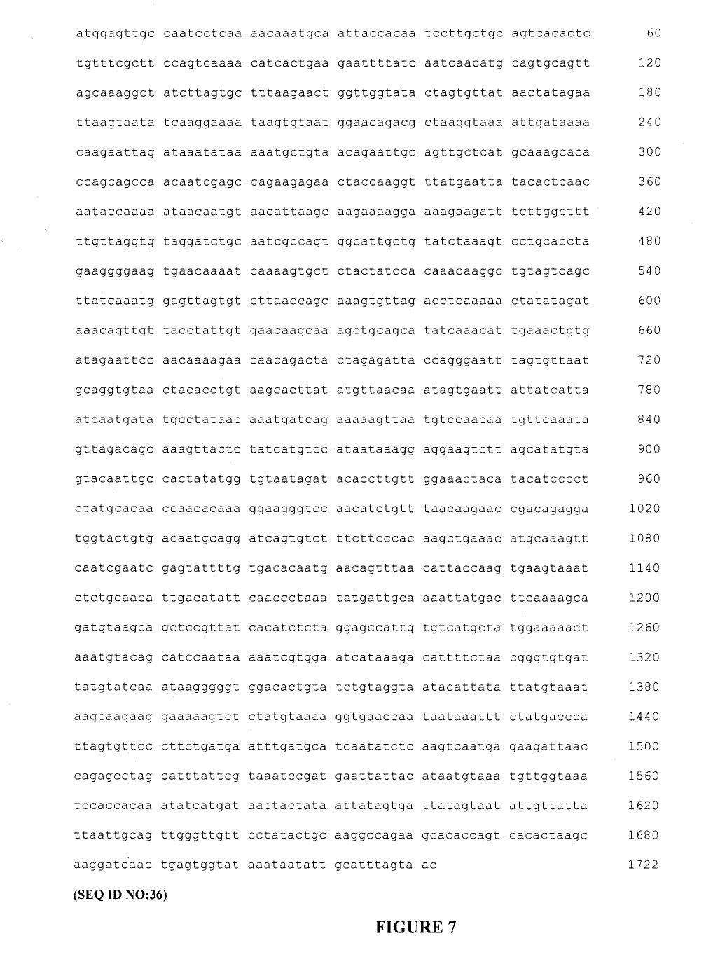

An isolated polynucleotide encoding the RSV G polypeptide of SEQ ID NO: 3 (amino acids 151-190) comprises the nucleotide sequence of SEQ ID NO: 10 (FIG. 6A). A codon-optimized polynucleotide encoding the RSV G polypeptide of SEQ ID NO:3, suitable for use in insect host cells, comprises the nucleotide sequence of SEQ ID NO:11 (FIG. 6B).

An isolated polynucleotide encoding the RSV G polypeptide of SEQ ID NO:4 (amino acids 151-186) comprises the nucleotide sequence of SEQ ID NO: 12 (FIG. 6C). A codon-optimized polynucleotide encoding the RSV G polypeptide of SEQ ID NO:4, suitable for use in insect host cells, comprises the nucleotide sequence of SEQ ID NO: 13 (FIG. 6D).

An isolated polynucleotide encoding the RSV G polypeptide of SEQ ID NO:5 (amino acids 151-172) comprises the nucleotide sequence of SEQ ID NO: 14 (FIG. 6E). A codon-optimized polynucleotide encoding the RSV G polypeptide of SEQ ID NO:5, suitable for use in insect host cells, comprises the nucleotide sequence of SEQ ID NO: 15 (FIG. 6F).

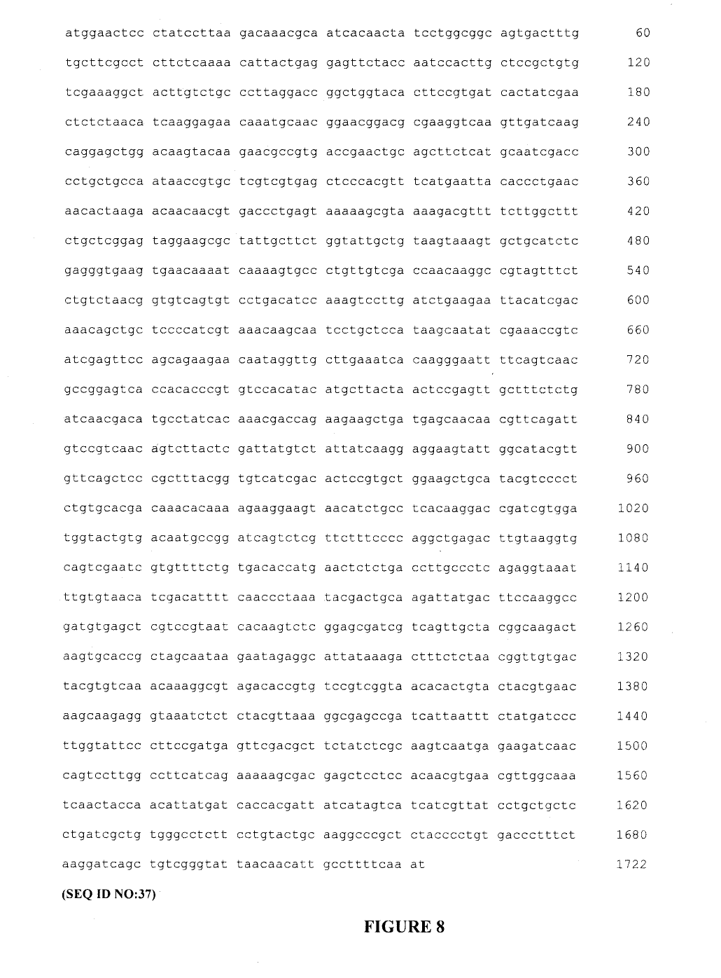

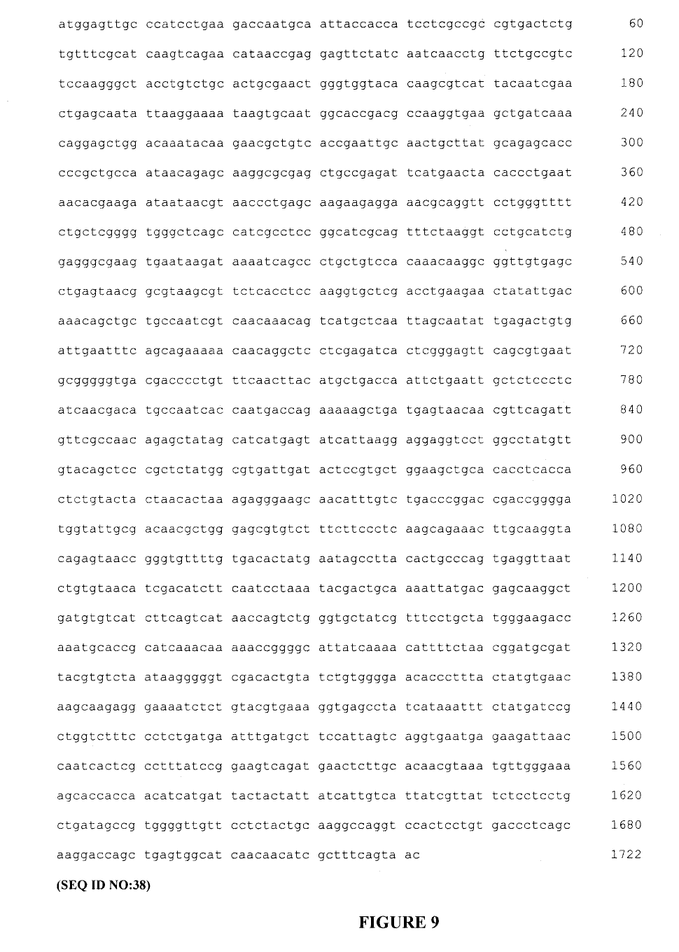

An isolated polynucleotide encoding the amino acid sequence of the RGH strain of RSV F (SEQ ID NO:2) comprises the nucleotide sequence of SEQ ID NO:36 (FIG. 7). A codon-optimized polynucleotide encoding the RGH RSV F amino acid of SEQ ID NO:2, suitable for use in insect host cells, comprises the nucleotide sequence of SEQ ID NO:37 (FIG. 8). A codon-optimized polynucleotide encoding the RGH RSV F amino acid sequence of SEQ ID NO:2, suitable for use in mammalian host cells, comprises the nucleotide sequence of SEQ ID NO:38 (FIG. 9).

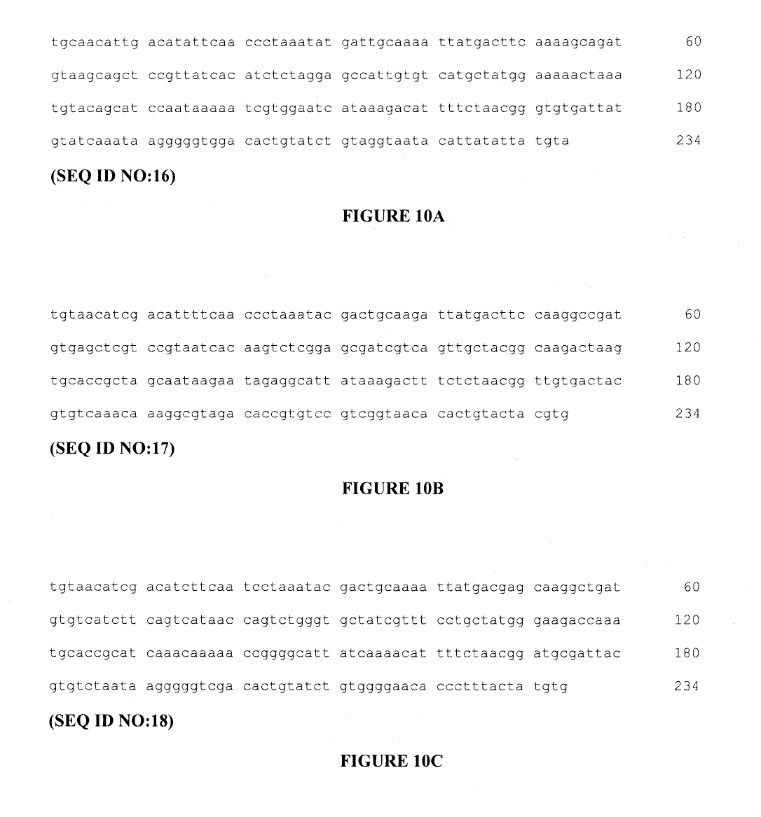

An isolated polynucleotide encoding the RSV F polypeptide of SEQ ID NO:7 (amino acids 382-459) comprises the nucleotide sequence of SEQ ID NO:16 (FIG. 10A). Codon-optimized polynucleotides encoding the RSV F polypeptide of SEQ ID NO:7, suitable for use in insect and mammalian host cells, comprise the nucleotide sequences of SEQ ID NOS: 17 (FIG. 10B) and 18 (FIG. 10C), respectively.

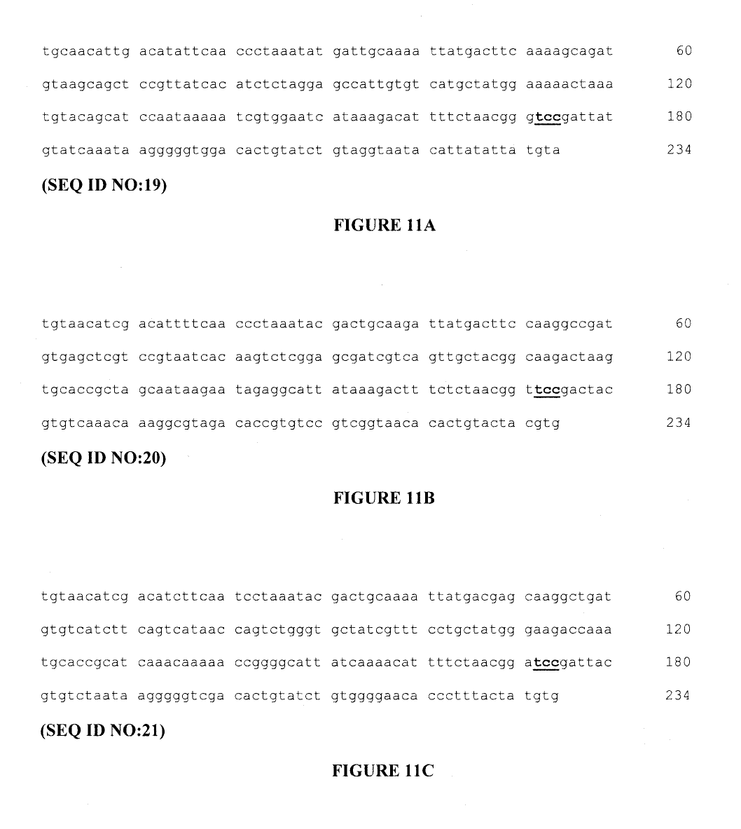

As discussed supra, the isolated RSV F polypeptide of SEQ ID NO:7 can optionally have a cysteine to serine substitution at amino acid position 58. An isolated polynucleotide encoding the RSV F polypeptide of SEQ ID NO: 7 containing this cysteine to serine substitution comprises the nucleotide sequence of SEQ ID NO:19 (FIG. 11A). Codon-optimized polynucleotides encoding the RSV F polypeptide of SEQ ID NO:7 containing the cysteine to serine substitution suitable for use in insect and mammalian host cells comprise the nucleotide sequences of SEQ ID NOS: 20 (FIG. 11B) and 21 (FIG. 11C), respectively.

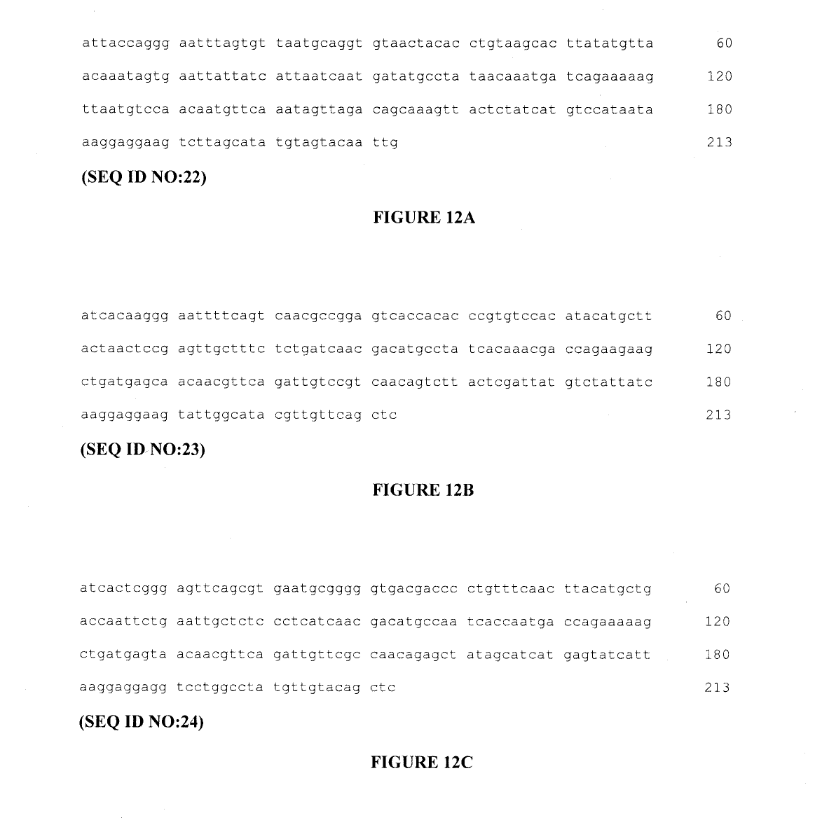

An isolated polynucleotide encoding the RSV F polypeptide of SEQ ID NO:8 (amino acids 233-303) comprises the nucleotide sequence of SEQ ID NO:22 (FIG. 12A). Codon-optimized polynucleotides encoding the RSV F polypeptide of SEQ ID NO:8, suitable for use in insect and mammalian host cells, comprise the nucleotide sequences of SEQ ID NOS: 23 (FIG. 12B) and 24 (FIG. 12C), respectively.

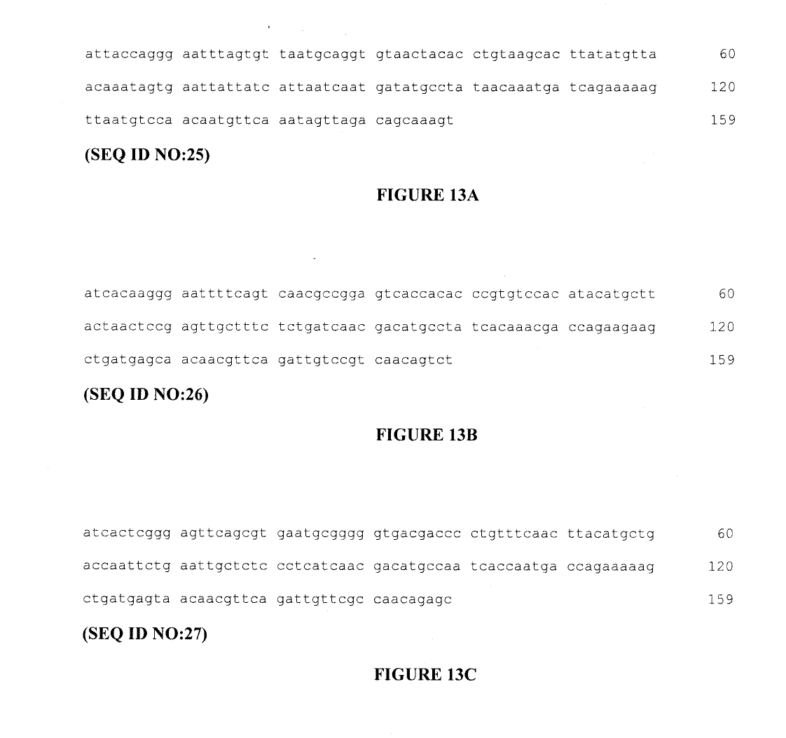

An isolated polynucleotide encoding the RSV F polypeptide of SEQ ID NO:9 (amino acids 233-286) comprises the nucleotide sequence of SEQ ID NO:25 (FIG. 13A). Codon-optimized polynucleotides encoding the RSV F polypeptide of SEQ ID NO:9, suitable for use in insect and mammalian host cells, comprise the nucleotide sequences of SEQ ID NOS: 26 (FIG. 13B) and 27 (FIG. 13C), respectively.

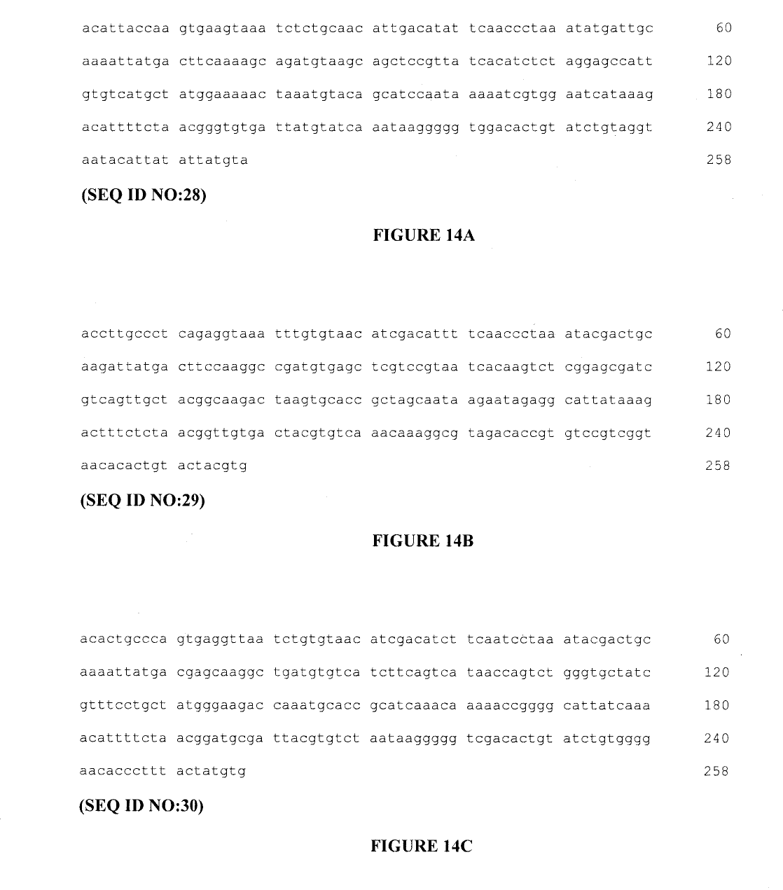

An isolated polynucleotide encoding the RSV F polypeptide of SEQ ID NO:6 (amino acids 374-459) comprises the nucleotide sequence of SEQ ID NO:28 (FIG. 14A). Codon-optimized polynucleotides encoding the RSV F polypeptide of SEQ ID NO:6, suitable for use in insect and mammalian host cells, comprise the nucleotide sequences of SEQ ID NOS: 29 (FIG. 14B) and 30 (FIG. 14C), respectively.

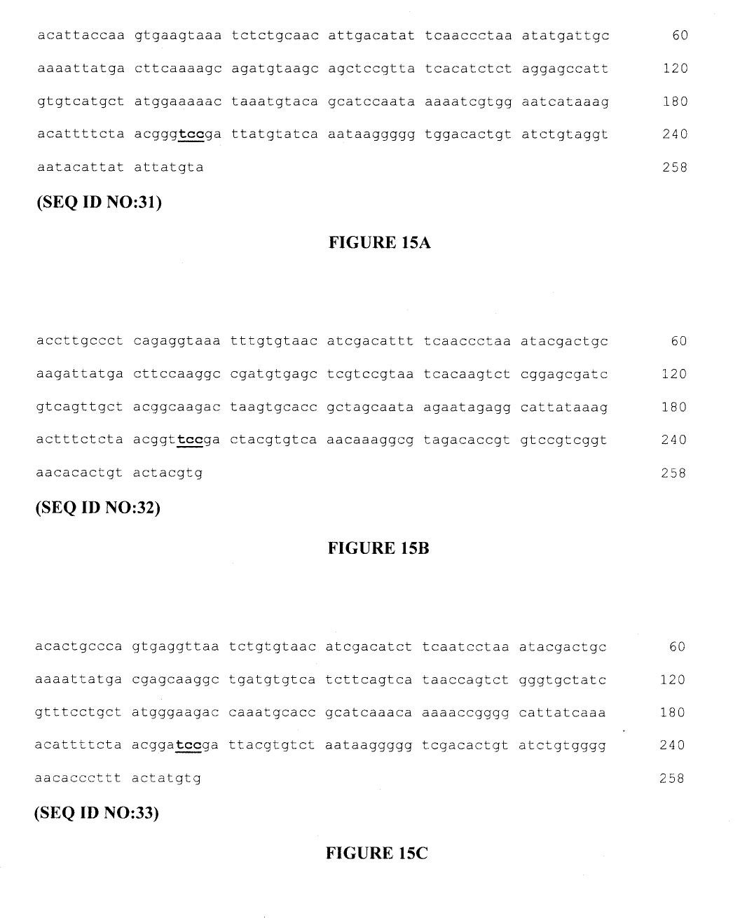

As discussed supra, the isolated RSV F polypeptide of SEQ ID NO:6 can optionally have a cysteine to serine substitution at amino acid position 66. An isolated polynucleotide encoding the RSV F polypeptide of SEQ ID NO: 6 containing this cysteine to serine substitution comprises the nucleotide sequence of SEQ ID NO:31 (FIG. 15A). Codon-optimized polynucleotides encoding the RSV F polypeptide of SEQ ID NO:6 containing the cysteine to serine substitution suitable for use in insect and mammalian host cells comprise the nucleotide sequences of SEQ ID NOS: 32 (FIG. 15B) and 33 (FIG. 15C), respectively.

Another aspect of the present invention relates to a recombinant transgene that includes any one of the polynucleotide sequences of the present invention, including the polynucleotides of SEQ ID NOS:10-38 and the polynucleotides encoding the RSV G and RSV F fusion proteins, operably coupled to a promoter-effective DNA molecule, a leader DNA sequence comprising a start-codon, and a transcription termination sequence. Selection of a suitable promoter-effective DNA molecule and other components of the recombinant transgene should be tailored to the expression system and host cell used to facilitate expression. A number of suitable promoter molecules are described infra.

Another aspect of the present invention is directed to a recombinant vector comprising any one of the above described polynucleotides or recombinant transgenes of the present invention. In accordance with this aspect of the present invention, the recombinant vector can contain any of the isolated polynucleotides of SEQ ID NOS: 10-38, the polynucleotides encoding the RSV G and RSV F fusion proteins, or the above described recombinant transgenes.

In accordance with this aspect of the invention, the polynucleotides of the present invention are inserted into an expression system or vector to which the molecule is heterologous. The heterologous nucleic acid molecule is inserted into the expression system or vector in proper sense (5′→3′) orientation relative to the promoter and any other 5′ regulatory molecules, and correct reading frame. The preparation of the nucleic acid constructs can be carried out using standard cloning methods well known in the art as described by SAMBROOK AND RUSSELL, MOLECULAR CLONING: A LABORATORY MANUAL (Cold Springs Laboratory Press, 2001), which is hereby incorporated by reference in its entirety. U.S. Pat. No. 4,237,224 to Cohen and Boyer, which is hereby incorporated by reference in its entirety, also describes the production of expression systems in the form of recombinant plasmids using restriction enzyme cleavage and ligation with DNA ligase.

Suitable expression vectors include those which contain replicon and control sequences that are derived from species compatible with the host cell. For example, if E. coli is used as a host cell, plasmids such as pUC19, pUC18 or pBR322 may be used. When using insect host cells, appropriate transfer vectors compatible with insect host cells include, pVL1392, pVL1393, pAcGP67 and pAcSecG2T, which incorporate a secretory signal fused to the desired protein, and pAcGHLT and pAcHLT, which contain GST and 6×His tags (BD Biosciences, Franklin Lakes, N.J.). Viral vectors suitable for use in carrying out this aspect of the invention include, adenoviral vectors, adeno-associated viral vectors, vaccinia viral vectors, nodaviral vectors, and retroviral vectors. Other suitable expression vectors are described in SAMBROOK AND RUSSELL, MOLECULAR CLONING: A LABORATORY MANUAL (Cold Springs Laboratory Press, 2001), which is hereby incorporated by reference in its entirety. Many known techniques and protocols for manipulation of nucleic acids, for example in preparation of nucleic acid constructs, mutagenesis, sequencing, introduction of DNA into cells and gene expression, and analysis of proteins, are described in detail in CURRENT PROTOCOLS IN MOLECULAR BIOLOGY (Fred M. Ausubel et al. eds., 2003), which is hereby incorporated by reference in its entirety.

Different genetic signals and processing events control many levels of gene expression (e.g., DNA transcription and messenger RNA (“mRNA”) translation) and subsequently the amount of RSV G and RSV F polypeptides and fusion proteins that are produced and expressed by the host cell. Transcription of DNA is dependent upon the presence of a promoter, which is a DNA sequence that directs the binding of RNA polymerase, and thereby promotes mRNA synthesis. Promoters vary in their “strength” (i.e., their ability to promote transcription). For the purposes of expressing a cloned gene, it is desirable to use strong promoters to obtain a high level of transcription and, hence, expression. Depending upon the host system utilized, any one of a number of suitable promoters may be used. For instance, when using E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene. When using insect cells, suitable baculovirus promoters include late promoters, such as 39K protein promoter or basic protein promoter, and very late promoters, such as the p10 and polyhedron promoters. In some cases it may be desirable to use transfer vectors containing multiple baculoviral promoters.

Translation of mRNA in prokaryotes depends upon the presence of the proper prokaryotic signals, which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and promote binding of mRNA to ribosomes by duplexing with the rRNA to allow correct positioning of the ribosome. For a review on maximizing gene expression, see Roberts and Lauer, “Maximizing Gene Expression on a Plasmid Using Recombination In Vitro,” Methods in Enzymology, 68:473-82 (1979), which is hereby incorporated by reference in its entirety.

Host cells suitable for expressing the RSV G or RSV F polypeptides, fusion proteins, or recombinant transgenes include any one of the more commonly available gram negative bacteria. Suitable microorganisms include Pseudomonas aeruginosa, Escherichia coli, Salmonella gastroenteritis (typhimirium), S. typhi, S. enteriditis, Shigella flexneri, S. sonnie, S. dysenteriae, Neisseria gonorrhoeae, N. meningitides, Haemophilus influenzae, H. pleuropneumoniae, Pasteurella haemolytica, P. multilocida, Legionella pneumophila, Treponema pallidum, T. denticola, T. orales, Borrelia burgdorferi, Borrelia spp., Leptospira interrogans, Klebsiella pneumoniae, Proteus vulgaris, P. morganii, P. mirabilis, Rickettsia prowazeki, R. typhi, R. richettsii, Porphyromonas (Bacteriodes) gingivalis, Chlamydia psittaci, C. pneumoniae, C. trachomatis, Campylobacter jejuni, C. intermedis, C. fetus, Helicobacter pylori, Francisella tularenisis, Vibrio cholerae, Vibrio parahaemolyticus, Bordetella pertussis, Burkholderie pseudomallei, Brucella abortus, B. susi, B. melitens is, B. canis, Spirillum minus, Pseudomonas mallei, Aeromonas hydrophile, A. salmonicida, and Yersinia pestis.

In addition to bacteria cells, animal cells, in particular mammalian and insect cells, yeast cells, fungal cells, plant cells, or algal cells are also suitable host cells for transfection/transformation of the recombinant expression vector carrying an isolated polynucleotide molecule of the present invention. Mammalian cell lines commonly used in the art include Chinese hamster ovary cells, HeLa cells, baby hamster kidney cells, COS cells, and many others. Suitable insect cell lines include those susceptible to baculoviral infection, including Sf9 and Sf21 cells.

Methods for transforming/transfecting host cells with expression vectors are well-known in the art and depend on the host system selected, as described in SAMBROOK AND RUSSELL, MOLECULAR CLONING: A LABORATORY MANUAL (Cold Springs Laboratory Press, 2001), which is hereby incorporated by reference in its entirety. For bacterial cells, suitable techniques include calcium chloride transformation, electroporation, and transfection using bacteriophage For eukaryotic cells, suitable techniques include calcium phosphate transfection, DEAE-Dextran, electroporation, liposome-mediated transfection, and transduction using retrovirus or any other viral vector. For insect cells, the transfer vector containing the polynucleotide construct of the present invention is co-transfected with baculovirus DNA, such as AcNPV, to facilitate the production of a recombinant virus resulting from homologous recombination between the RSV G or RSV F polynucleotide construct in the transfer vector and baculovirus DNA. Subsequent recombinant viral infection of Sf cells results in a high rate of recombinant protein production. Regardless of the expression system and host cell used to facilitate protein production, the expressed polypeptides and fusion proteins of the present invention can be readily purified using standard purification methods known in the art and described in PHILIP L. R. BONNER, PROTEIN PURIFICATION (Routledge 2007), which is hereby incorporated by reference in its entirety.

The present invention is also directed to isolated antibodies having antigen specificity for the one or more neutralizing epitopes of the RSV G or RSV F polypeptides of the present invention.

The isolated antibodies of the present invention may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. The isolated antibody can be a full length antibody, monoclonal antibody (including full length monoclonal antibody), polyclonal antibody, multispecific antibody (e.g., bispecific antibody), human, humanized or chimeric antibody, and antibody fragments, e.g., Fab fragments, F(ab′) fragments, fragments produced by a Fab expression library, epitope-binding fragments of any of the above, and engineered forms of antibodies, e.g., scFv molecules, so long as they exhibit the desired activity, e.g., RSV G or RSV F neutralizing activity.

Polyclonal antibodies can be prepared by any method known in the art. Polyclonal antibodies can be raised by immunizing an animal (e.g., a rabbit, rat, mouse, donkey, etc.) with multiple subcutaneous or intraperitoneal injections of the relevant antigen, e.g., an isolated RSV G or RSV F polypeptide fragment, RSV G or F fusion protein, or immunogenic conjugate) diluted in sterile saline and combined with an adjuvant (e.g., Complete or Incomplete Freund's Adjuvant) to form a stable emulsion. The polyclonal antibody is then recovered from blood or ascites of the immunized animal. Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, etc. Polyclonal antiserum can also be rendered monospecific using standard procedures (see e.g., Agaton et al., “Selective Enrichment of Monospecific Polyclonal Antibodies for Antibody-Based Proteomics Efforts,” J Chromatography A 1043(1):33-40 (2004), which is hereby incorporated by reference in its entirety).

Monoclonal antibodies can be prepared using hybridoma methods, such as those described by Kohler and Milstein, “Continuous Cultures of Fused Cells Secreting Antibody of Predefined Specificity,” Nature 256:495-7 (1975), which is hereby incorporated by reference in its entirety. Using the hybridoma method, a mouse, hamster, or other appropriate host animal, is immunized to elicit the production by lymphocytes of antibodies that will specifically bind to an immunizing antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against RSV G or RSV F, as determined by immunoprecipitation, immunoblotting, or by an in vitro binding assay such as radioimmunoassay (RIA) or enzyme-linked immunosorbent assay (ELISA) can then be propagated either in in vitro culture using standard methods (JAMES W. GODING, MONOCLONAL ANTIBODIES: PRINCIPLES AND PRACTICE (Academic Press 1986), which is hereby incorporated by reference in its entirety) or in vivo as ascites tumors in an animal. The monoclonal antibodies can then be purified from the culture medium or ascites fluid as described for polyclonal antibodies above.

Alternatively monoclonal antibodies can also be made using recombinant DNA methods as described in U.S. Pat. No. 4,816,567 to Cabilly et al, which is hereby incorporated by reference in its entirety. Polynucleotides encoding a monoclonal antibody are isolated, from mature B-cells or hybridoma cell, by RT-PCR using oligonucleotide primers that specifically amplify the genes encoding the heavy and light chains of the antibody. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries as described (McCafferty et al., “Phage Antibodies: Filamentous Phage Displaying Antibody Variable Domains,” Nature 348:552-554 (1990); Clackson et al., “Making Antibody Fragments Using Phage Display Libraries,” Nature, 352:624-628 (1991); and Marks et al., “By-passing Immunization. Human Antibodies from V-gene Libraries Displayed on Phage,” J Mol Biol 222:581-597 (1991), which are hereby incorporated by reference in their entirety).

The polynucleotide(s) encoding a monoclonal antibody can further be modified in a number of different ways using recombinant DNA technology to generate alternative antibodies. In one embodiment, the constant domains of the light and heavy chains of, for example, a mouse monoclonal antibody can be substituted for those regions of a human antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

Another aspect of the present invention is directed to a pharmaceutical composition. This pharmaceutical composition contains any one of the isolated RSV G or RSV F polypeptides, any one of the RSV G or RSV F fusion proteins, or the RSV G or RSV F immunogenic conjugates of the present invention. The pharmaceutical composition can alternatively contain any one of the polynucleotides or the recombinant transgene of the present invention encoding any of the isolated RSV G or RSV F polypeptides or fusions proteins described above. These agents can be used to generate immunity in a recipient.

Alternatively, the present invention also relates to a pharmaceutical composition that includes an antibody of the present invention. This type of composition can be used to afford passive immunity against RSV in a recipient.

The pharmaceutical compositions of the present invention also contain a pharmaceutically acceptable carrier. Acceptable pharmaceutical carriers include solutions, suspensions, emulsions, excipients, powders, or stabilizers. The carrier should be suitable for the desired mode of delivery, discussed infra.

Pharmaceutical compositions suitable for injectable use (e.g., intravenous, intra-arterial, intramuscular, etc.) may include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. Suitable adjuvants, carriers and/or excipients, include, but are not limited to sterile liquids, such as water and oils, with or without the addition of a surfactant and other pharmaceutically and physiologically acceptable carriers. Illustrative oils are those of petroleum, animal, vegetable, or synthetic origin, for example, peanut oil, soybean oil, or mineral oil. In general, water, saline, aqueous dextrose and related sugar solutions, and glycols, such as propylene glycol or polyethylene glycol, are preferred liquid carriers, particularly for injectable solutions.

Oral dosage formulations can include standard carriers such as pharmaceutical grades of mannitol, lactose, starch, magnesium stearate, sodium saccharine, cellulose, magnesium carbonate, etc. Suitable carriers include lubricants and inert fillers such as lactose, sucrose, or cornstarch. In another embodiment, these compounds are tableted with conventional tablet bases such as lactose, sucrose, or cornstarch in combination with binders like acacia, gum gragacanth, cornstarch, or gelatin; disintegrating agents such as cornstarch, potato starch, or alginic acid; a lubricant like stearic acid or magnesium stearate; sweetening agents such as sucrose, lactose, or saccharine; and flavoring agents such as peppermint oil, oil of wintergreen, or artificial flavorings. Generally, the ingredients are supplied either separately or mixed together in unit dosage form, for example, as a dry lyophilized powder or water free concentrate in a hermetically sealed container such as an ampule or sachette indicating the quantity of active agent.

Formulations suitable for transdermal delivery can also be prepared in accordance with the teachings of Lawson et al., “Use of Nanocarriers for Transdermal Vaccine Delivery,” Clin Pharmacol Ther 82(6):641-3 (2007), which is hereby incorporated by reference in its entirety.

Formulations suitable for intranasal nebulization or bronchial aerosolization delivery are also known and can be used in the present invention (see Lu & Hickey, “Pulmonary Vaccine Delivery,” Exp Rev Vaccines 6(2):213-226 (2007) and Alpar et al., “Biodegradable Mucoadhesive Particulates for Nasal and Pulmonary Antigen and DNA Delivery,” Adv Drug Deliv Rev 57(3):411-30 (2005), which are hereby incorporated by reference in their entirety.

The pharmaceutical compositions of the present invention can also include an effective amount of an adjuvant. In pharmaceutical compositions containing a fusion protein, an additional, preferably distinct adjuvant is included in the composition. Suitable adjuvants include, without limitation, Freund's complete or incomplete, mineral gels such as aluminum hydroxide, surface active substances such as lysolecithin, pluronic polyols, polyanions, peptides, oil emulsions, dinitrophenol, and potentially useful human adjuvants such as Bacille Calmette-Guerin, Carynebacterium parvum, non-toxic Cholera toxin, flagellin, iscomatrix, and liposome polycation DNA particles.

The present invention also relates to a method of inducing a neutralizing immune response against RSV in a subject. This method involves administering to the subject an RSV G or RSV F polypeptide or fusion peptide of the present invention or a pharmaceutical composition comprising the same in an amount effective to induce a neutralizing immune response against RSV

It is contemplated that the individual to be treated in accordance with the present invention can be any mammal, but preferably a human. Veterinary uses are also contemplated. While the individual can be any mammal that is known to be susceptible to RSV infection; the RSV polypeptide, RSV fusion protein, RSV immunogenic conjugate, or the pharmaceutical composition containing the same is preferably derived from a genotype that is specific to the host mammal to be immunized in accordance with the present invention. For example, for inducing an immune response in humans it is preferable that the RSV polypeptide is derived from a human RSV strain. Moreover, the pharmaceutical composition can be multi-valent, containing antigen directed to different RSV strains, which collectively provide a more protective immune response. The individual to be treated is preferably an infant or juvenile, an elderly individual, or an individual having a cardiopulmonary or immunosuppressive condition.

Effective amounts of the composition used to induce an immune response against RSV will depend upon the mode of administration, frequency of administration, nature of the treatment, age and condition of the individual to be treated, and the type of pharmaceutical composition used to deliver the compound. Effective levels of the composition may range from about 0.001 to about 2.5 mg/kg depending upon the clinical endpoints and toxicity thresholds. While individual doses may vary, optimal ranges of the effective amounts may be determined by one of ordinary skill in the art.

The pharmaceutical composition can be administered by any means suitable for producing the desired immune response. Preferred delivery routes include orally, by inhalation, by intranasal instillation, topically, transdermally, parenterally, subcutaneously, intravenous injection, intra-arterial injection, intramuscular injection, intraplurally, intraperitoneally, or by application to mucous membrane. The composition can be delivered repeatedly over a course of time, i.e., according to a prime/boost regiment, that achieves optimal enhancement of the immune response.

The RSV G or RSV F polypeptide or fusion peptides of the present invention, and pharmaceutical compositions comprising the same can be incorporated into a delivery vehicle to facilitate administration. Such delivery vehicles include, but are not limited to, biodegradable microspheres (MARK E. KEEGAN & W. MARK SALTZMAN, Surface Modified Biodegradable Microspheres for DNA Vaccine Delivery, in DNA VACCINES: METHODS AND PROTOCOLS 107-113 (W. Mark Saltzman et al., eds., 2006), which is hereby incorporated by reference in its entirety), microparticles (Singh et al., “Nanoparticles and Microparticles as Vaccine Delivery Systems,” Expert Rev Vaccine 6(5):797-808 (2007), which is hereby incorporated by reference in its entirety), nanoparticles (Wendorf et al., “A Practical Approach to the Use of Nanoparticles for Vaccine Delivery,” J Pharmaceutical Sciences 95(12):2738-50 (2006) which is hereby incorporated by reference in its entirety), liposomes (U.S. Patent Application Publication No. 20070082043 to Dov et al. and Hayashi et al., “A Novel Vaccine Delivery System Using Immunopotentiating Fusogenic Liposomes,” Biochem Biophys Res Comm 261(3): 824-28 (1999), which are hereby incorporated by reference in their entirety), collagen minipellets (Lofthouse et al., “The Application of Biodegradable Collagen Minipellets as Vaccine Delivery Vehicles in Mice and Sheep,” Vaccine 19(30):4318-27 (2001), which is hereby incorporated by reference in it entirety), and cochleates (Gould-Fogerite et al., “Targeting Immune Response Induction with Cochleate and Liposome-Based Vaccines,” Adv Drug Deliv Rev 32(3):273-87 (1998), which is hereby incorporated by reference in its entirety).

The compositions of the present invention can further be formulated for the desired mode of administration. For example, the composition can be formulated into a single-unit oral dosage, an injectable dose contained in a syringe, a transdermally deliverable dosage contained in a transdermal patch, or an inhalable dose contained in an inhaler.

For prophylactic treatment against RSV infection, it is intended that the composition(s) of the present invention can be administered prior to exposure of an individual to the RSV and that the resulting immune response can inhibit or reduce the severity of the RSV infection such that the RSV can be eliminated from the individual. The pharmaceutical compositions of the present invention can also be administered to an individual for therapeutic treatment. In accordance with embodiment, it is intended that the composition(s) of the present invention can be administered to an individual who is already exposed to the RSV. The resulting enhanced immune response will reduce the duration or severity of the existing RSV infection, as well as minimize any harmful consequences of untreated RSV infections. The composition(s) can also be administered in combination other therapeutic anti-RSV regimen.

### EXAMPLES

The following examples are provided to illustrate embodiments of the present invention but are by no means intended to limit its scope.

### Materials and Methods for Examples 1-2

Mammalian Cells and RSV Isolates: The HEp-2 epidermoid laryngeal carcinoma cell line and RSV strains B1 and A2 were obtained from ATCC (Manassas, Va.). HEp-2 cells were maintained in Minimal Eagle's Media (MEM)+5% fetal calf serum and were used to propagate RSV as previously described (Walsh et al., “Comparison of Antigenic Sites of Subtype-specific Respiratory Syncytial Virus Attachment Proteins,” J Gen Virol 70 (Pt 11):2953-2961 (1989), which is hereby incorporated by reference in its entirety). The RGH RSV strain (subtype A5) is a clinical isolate that was collected during the 1999-2000 winter season (Falsey et al., “Respiratory Syncytial Virus Infection in Elderly Adults,” N Eng J Med 52:1749-59 (2005), which is hereby incorporated by reference in its entirety).

DNA Constructions and Manipulations: All restriction and DNA modification enzymes were obtained from New England Biolabs (NEB; Ipswich, Mass.). All oligonucleotides were purchased from Eurofins MWG Operon (Huntsville, Ala.).

For the isolation of full-length RGH strain RSV G cDNA, total RNA was isolated from RGH RSV strain-infected HEp-2 cells and used as the template in RT-PCR reactions. The following oligonucleotides bearing the BamHI site (bolded) near their respective 5′ termini were used in the amplification processes: 5′GCGGATCCCCAAGAGCTCGAGTCAATACATAG3′ (SEQ ID NO:39) (including the cDNA sequence encoding the carboxy-terminus of the prototypical RSV subtype A SH protein) and 5′GGGGATCCGACTGCAGCAAGGATTGTGG3′ (SEQ ID NO:40) (complementary to nucleotides 34-53 of the cDNA encoding the previously isolated RGH strain RSV F protein (GenBank accession ABI35685.1)) (Chen et al., “Conservation of the Respiratory Syncytial Virus SH Gene,” J Infect Dis 182:1228-1233 (2000), which is hereby incorporated by reference in its entirety). Access RTPCR system (Promega; Madison, Wis.) was used according to the manufacturer's instructions and the reaction conditions were as follows: 45° C.×45 min for reverse transcriptase-mediated first strand synthesis; 94° C.×2 min for initial denaturation; 35 amplification cycles of 94° C.×30 sec, 60° C.×1 min, 68° C.×2 min; final extension at 68° C.×7 min; and overnight storage at 4° C. The resulting ˜1,000 bp amplicon was ligated into pCR2.1 (Invitrogen, Carlsbad, Calif.) and both strands were sequenced and codon optimized (DNA2.0, Menlo Park, Calif.).

For generation of plasmids directing the synthesis of glutathione S-transferase (GST)-RSV G fusion proteins, two construction strategies were employed. For GST-G151-190 (unless otherwise specified, the RSV G-derived residues are derived from the RGH strain G protein), oligonucleotides complementary to the appropriate codons, bearing the sequence 5′GCGGATCC (SEQ ID NO:41) (BamHI site bolded) and encoding a termination codon immediately following RSV G codon 190 were utilized in a standard PCR reaction (PCR Supermix High Fidelity Taq; Invitrogen) using the codon-optimized RGH G cDNA as the template. The resulting ˜120 bp amplicon was purified, digested sequentially with BamHI and DpnI (to remove the plasmid template) and ligated into the BamHI site of pGEX4T-1 (Amersham Biosciences, Pittsburgh, Pa.). For plasmid constructions encoding shorter fragments of RSV G protein or residues 151-172 from the A2 or B1 strains, complementary oligonucleotide pairs encoding the relevant G amino acid residues from A2, B1, or RGH strains, bearing BamHI-cohesive ends, and a termination codon after the last G-derived codon were treated with T4 kinase, annealed, and ligated into the BamHI restriction enzyme sites of pGEX4T-1 (Johnson et al., “The G Glycoprotein of Human Respiratory Syncytial Viruses of Subgroups A and B: Extensive Sequence Divergence Between Antigenically Related Proteins,” Proc Natl Acad Sci U.S.A. 84:5625-5629 (1987) and Wertz et al., “Nucleotide Sequence of the G Protein Gene of Human Respiratory Syncytial Virus Reveals an Unusual Type of Viral Membrane Protein,” Proc Natl Acad Sci U.S.A. 82:4075-4079 (1985), which are hereby incorporated by reference in their entirety). The RSV G cDNA-derived inserts were verified by dideoxy sequencing (ACGT Inc., Wheeling, Ill.). Unless otherwise specified, the DH5α E. coli strain (EMD Chemicals, Gibbstown, N.J.) was used for routine plasmid manipulations.

Bacterial Manipulations and Protein Purification: For plasmid and bacterial selections, carbenicillin (Sigma-Aldrich, St. Louis, Mo.), with or without tetracycline and kanamycin (both from EMD Chemicals) were used as deemed appropriate for the bacterial and plasmid markers. Miller's LB broth base (Invitrogen) and Bacto-Agar (BD, Franklin Lakes, N.J.) were routinely used for bacterial propagation.

Each of the pGEX4T-1 derivatives were transformed into the DH5α E. coli strain and the recombinant GST-fusion proteins were expressed using 0.1-0.2 mM isopropyl β-D-1-thiogalactopyranoside (IPTG; Invitrogen) and purified according to the plasmid manufacturer's recommendations (Amersham). To ensure that the appropriate disulfide bonds are formed within the G loop region, GST-G173-190 was synthesized and purified from both DH5α and Origami 2 (EMD Chemicals); in the latter bacterial strain, disulfide bond formation of heterologous proteins is favored due to mutations in the thioredoxin and glutathione reductases (Lauber et al., “Accurate Disulfide Formation in Escherichia coli: Overexpression and Characterization of the First Domain (HF6478) of the Multiple Kazal-type Inhibitor LEKTI,” Protein Expr Purif 22:108-112 (2001), which is hereby incorporated by reference in its entirety).

The subtype A-specific RSV G protein was purified from RSV A2-infected HEp2 cells as previously described (Walsh et al., “Purification and Characterization of GP90, One of the Envelope Glycoproteins of Respiratory Syncytial Virus,” J Gen Virol 65 (Pt 4):761-767 (1984), which is hereby incorporated by reference in its entirety).

For protein gel electrophoresis, bacterially derived proteins (typically 0.5-1 μg) or purified RSV G protein (typically 0.1-0.5 μg) was diluted 1:1 (vol:vol) with 2×SDS-sample buffer containing β-mercaptoethanol (Sigma-Aldrich), heated at 95° C. for 2-5 minutes, and then resolved on 12%/6% discontinuous SDS-PAGE using the Protean Tetra-cell apparatus (Bio-Rad, Hercules, Calif.). Where appropriate, prestained molecular weight (MW) markers (Novex; Invitrogen) were resolved in parallel and proteins were visualized by staining with Coomassie Brilliant Blue R-250 (Bio-Rad).

Immunological Assays and Graphical/Statistical Analyses: The anti-RSV G mAbs L9, which was generated by E. E. Walsh, and K6, which was a gift to E. E. Walsh from P. Paradiso (Wyeth, Pearl River, N.Y.), have previously been characterized with respect to their RSV subtype-independent neutralization activities and reactogenicity against purified G protein in immunoblots and ELISAs (Walsh et al., “Comparison of Antigenic Sites of Subtype-specific Respiratory Syncytial Virus Attachment Proteins,” J Gen Virol 70 (Pt 11):2953-2961 (1989) and Walsh et al., “Purification and Characterization of GP90, One of the Envelope Glycoproteins of Respiratory Syncytial Virus,” J Gen Virol 65 (Pt 4):761-767 (1984), which are hereby incorporated by reference in their entirety).

For immunoblots, Magic Mark (Invitrogen) MW standards and purified, bacterially derived proteins were resolved on 12/6% SDS-PAGE gels and then transferred onto nitrocellulose using a BioRad Trans-blot device (typically at 100V at room temperature for 1 hr). PBST (Phosphate buffered saline (PBS) pH 7.4+0.1% Tween-20 (both from EMD Chemicals))+2% dried non-fat milk was used to block non-specific protein binding onto nitrocellulose. For detection of purified RSV G protein and bacterially derived RSV G derivatives, L9 or K6 was routinely used at 1:5,000 or 1:10,000 dilution in PBST followed by goat anti-mouse IgG heavy/light chain antibody-horseradish peroxidase (HRP) conjugate (Southern Biotech, Birmingham, Ala.) at 1:20,000 dilution in PBST. Antibody-antigen complexes were visualized by chemiluminescence (ECL; Thermo Scientific, Rockford, Ill.) and radiography (Kodak, Rochester, N.Y.).

Enzyme-linked immunosorbent assays (ELISAs) were performed essentially as previously described (Murata et al., “Antigenic Presentation of Heterologous Epitopes Engineered into the Outer Surface-Exposed Helix 4 Loop Region of Human Papillomavirus L1 Capsomeres,” Virol J 6:81 (2009) and Walsh et al., “Comparison of Antigenic Sites of Subtype-specific Respiratory Syncytial Virus Attachment Proteins,” J Gen Virol 70 (Pt 11):2953-2961 (1989), which are hereby incorporated by reference in their entirety). Typically, each recombinant RSV G derivative or purified G protein was diluted in bicarbonate buffer pH 9.0 and plated (50-100 ng/well) onto 96 well ELISA plates (Nalgene Nunc, Rochester, N.Y.) and incubated overnight at 4° C. Following incubation with L9 or K6 mAbs (typically at 1:10,000 dilution in PBS/0.2% Tween-20) as described in figure legends, alkaline phosphatase-conjugated goat anti-mouse secondary antibodies (Southern Biotech) and phosphatase substrate tablets (Sigma-Aldrich) were then used to visualize antigen-antibody complexes. The resulting colorimetric reactions were read at OD₄₀₅ₙₘ using a 96-well ELISA plate reader (Molecular Devices, Sunnyvale, Calif.).

Graphical representation of ELISA data were performed using Excel 2003 (Microsoft, Redmond, Wash.). Statistical manipulations were performed using JMP version 8.0 (SAS). For univariate analyses, means were compared with Wilcoxon rank-sum tests and proportions were compared using two-tailed Fisher's exact tests.

Collection and Immunological Analysis of RSV Subtype A-Infected Adult Sera: As part of an institutional review board-approved, previously completed clinical study on the epidemiology of RSV among elderly and hospitalized adults, acute and convalescent sera were collected from RSV-infected adults who provided consent and given patient identifier-unlinked study numbers (Falsey et al., “Respiratory Syncytial Virus Infection in Elderly Adults,” N Eng J Med 52:1749-59 (2005), which is hereby incorporated by reference in its entirety). Among the study participants, the serological diagnosis of RSV was made if there was a ≧four-fold increase from acute to convalescent serum immunoglobulin G (IgG) titers (in reciprocal log₂ dilutions) to purified, subtype-specific RSV F and/or G glycoproteins as measured in ELISAs (Falsey et al., “Respiratory Syncytial Virus Infection in Elderly Adults,” N Eng J Med 52:1749-59 (2005), which is hereby incorporated by reference in its entirety).

For this study, a subset of archived, paired sera samples that were collected from patients diagnosed with RSV A infection while in hospital (n=32) or outpatient (n=19) settings were identified. Aliquots of each sera sample were used in ELISAs against GST-G151-172 (RGH strain) and the resulting serum titers were expressed as reciprocal log₂ dilutions.

### Example 1

### Identification of Overlapping Neutralizing Epitopes Within the Central Unglycosylated Domain of the RSV G Protein

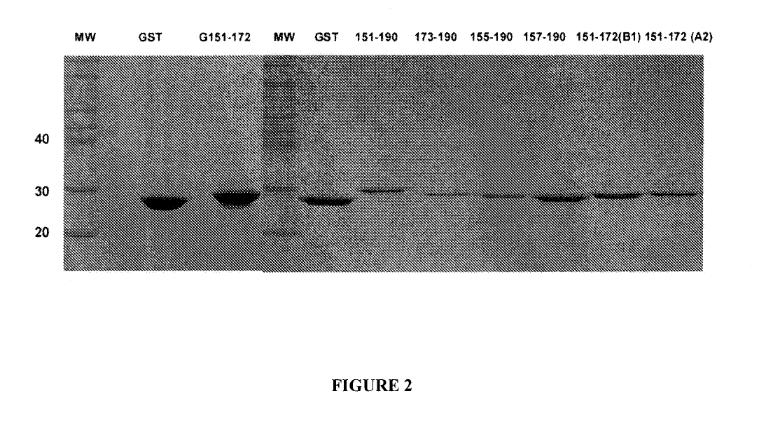

To determine the epitopes recognized by L9 and/or K6 mAbs, a series of pGEX-4T-1 derivatives were constructed, each programmed to synthesize either GST alone or GST-RSV G (RGH strain) fusion protein bearing a portion of the central unglycosylated region (amino acid residues 151-190 of SEQ ID NO:1 (SEQ ID NO:3)). These RSV G derived residues were subdivided into two subdomains: the “G stem” (amino acid residues 151-172 (SEQ ID NO:5)), and the “G loop” bearing amino acid residues 173-190 and its two cysteine disulfide bonds (FIG. 1A) (Melero et al., “Antigenic Structure, Evolution and Immunobiology of Human Respiratory Syncytial Virus Attachment (G) Protein,” J Gen Virol 78 (Pt 10):2411-2418 (1997) and Polack et al., “The Cysteine-rich Region of Respiratory Syncytial Virus Attachment Protein Inhibits Innate Immunity Elicited by the Virus and Endotoxin,” Proc Natl Acad Sci U.S.A. 102:8996-9001 (2005), which are hereby incorporated by reference in their entirety). GST-G proteins bearing amino- and carboxy-terminal truncations of the G stem region (e.g., residues 151-161 and 162-172) were generated as well as GST-G stem from RSV A2 and B1 strains. The resulting GST-G proteins were purified to >95% homogeneity (FIG. 2) and tested for recognition by L9 and K6 mAbs in immunoblots and ELISAs.

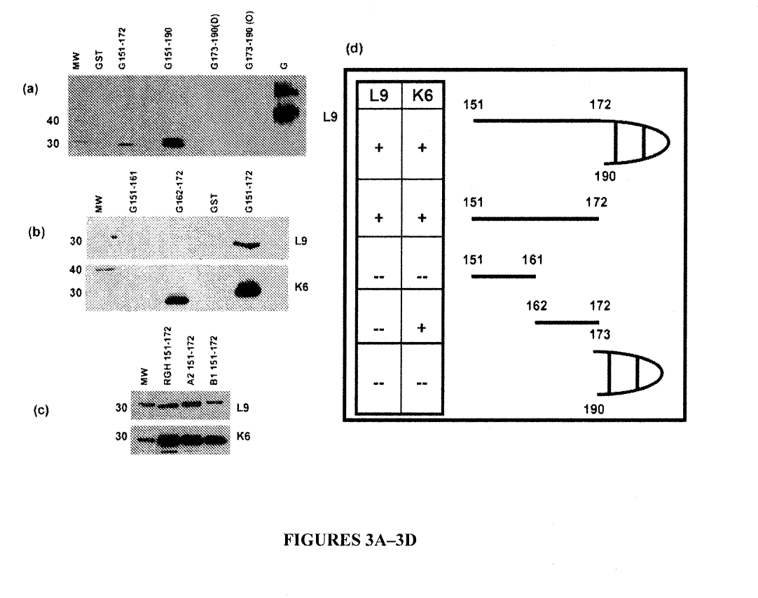

It was first determined whether the L9 epitope was present within the G stem and/or the G loop. In immunoblots, L9 recognized the purified, full-length G protein from the RSV A2 strain (positive control) as well as GST-G151-172, which contains the G stem region, and GST-G151-190, which bears the G stem+loop regions. In contrast, L9 did not bind to GST alone or to GST-G173-190 bearing the G loop region and synthesized in either DH5α or Origami 2 E. coli strains (FIG. 3A).

This panel of proteins was recognized in a similar manner by the K6 mAb. Crude bacterial extracts were generated bearing larger domains of RSV G fused to GST to ensure that there were no additional L9 or K6 epitopes outside the G stem-loop and within the RSV G ectodomain. Only extracts containing GST G67-190 (i.e., bearing the G stem-loop) were recognized by both mAbs while GST-G67-150 and G190-289 (i.e., portions of the G ectodomain that flank the central unglycosylated region) were non-reactogenic in immunoblots. Thus, the G stem residues 151-172 are sufficient for reactogenicity against L9 and K6 in immunoblots.

To better define the L9 and K6 binding sites and to ensure that the recognition of the G stem was not specific to RGH strain-derived residues, the binding of both mAbs to additional GST-G fusion proteins was tested. Neither mAb recognized GST-G151-161 bearing residues from the proximal half of the G stem region. In contrast, GST-G162-172 (i.e., the distal half of the G stem) was reproducibly detected by K6 but not by L9 (FIG. 3B). Furthermore, K6 but not L9 recognized amino-terminal truncations of G stem region as engineered into GST-G155-172 or GST-G157-172 proteins. Lastly, three variants of GST-G151-172, each bearing the corresponding residues from the RGH, A2, or B1 strains, were recognized by both mAbs (FIG. 3C). Taken together and as summarized in FIG. 3D, these data demonstrate that: 1) the full-length G stem is required for efficient recognition by L9 in immunoblots; 2) consistent with the subtype-independent neutralizing activity of both mAbs, K6 and L9 both recognized the G stem comprised of subtype A- and B-derived residues; and 3) the K6 epitope involves the distal 10 residues (162-172) of the G stem.

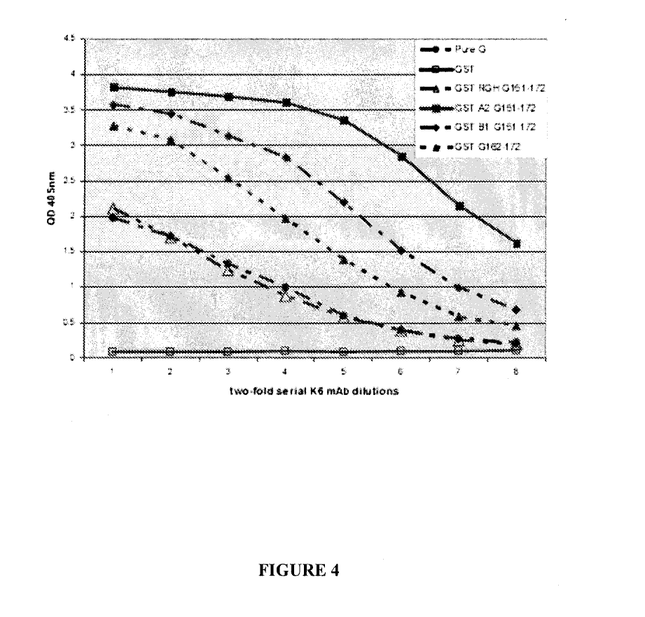

The binding of both mAbs to various GST-G derivatives was also examined under non-denaturing conditions in ELISAs. Consistent with immunoblot data, K6 recognized purified, RSV A2-derived G protein and GST-G151-172 (RGH strain) but not GST alone (FIG. 4). The interactions of K6 with GST-G stem derived from A2 and B1 strains were somewhat more robust than K6-GST G stem (RGH strain) binding (FIG. 4). L9 also exhibited slightly stronger interactions with GST-G151-172 from A2 and B1 strains as compared to those from RGH strain. Lastly, the reactogenicity of K6 with GST-G162-172 bearing the truncated G stem from the RGH strain was stronger than that bearing the full-length G stem (FIG. 4). Since the L9 and K6 mAb preparations were not directly compared against each other with respect anti-G immunoglobulin content and/or affinity, these data do not represent quantitative antibody-epitope binding measurements. However, the ELISA data confirm the binding of L9 and K6 mAbs to the G stem region assayed by immunoblots, and extend the binding data to non-denaturing conditions.

Taken together, these in vitro assays reveal that the epitopes of L9 and K6 mAbs are located within the G stem region and are overlapping. The L9 epitope is likely a longer epitope (aa 151-172) than that for K6 (aa 162-172); both epitopes are recognized by the corresponding mAbs under denaturing and non-denaturing conditions. Consistent with the RSV subtype-independent neutralizing activities of both mAbs, K6 and L9 both bound to GST fusion proteins bearing amino acids 151-172 from A2, B1, and RGH strains with varying qualitative degrees of variability in such reactogenicity in ELISAs. These biochemical epitope mapping results are in accord with genetic mutations induced by selective growth under the presence of L9 and other subtype-independent neutralizing mAbs.

### Example 2

### Reactogenicity of Human Sera from RSV-Infected Adults Against the RSV G Stem Region

Since the L9 and K6 epitopes are both localized within the G stem, the potential clinical and immunological relevance of these epitopes in human RSV infections was determined. To this end, the serum reactogenicity to GST-G151-172 was assayed in ELISAs using paired acute and convalescent sera from adults infected with RSV subtype A. These subjects were classified into one of two groups (hospitalized vs. outpatient) based on the initial location of patient screening (Falsey et al., “Respiratory Syncytial Virus Infection in Elderly Adults,” N Eng J Med 52:1749-59 (2005), which is hereby incorporated by reference in its entirety). In all paired sera, there was a ≧4-fold increase in the anti-RSV G (reciprocal log₂) titers from acute to convalescent sera.

Among paired sera from 32 RSV-infected hospitalized adults, 14 (44%) had a ≧4-fold increase in the anti-RSV G titers from acute to convalescent sera. Similarly, in serum samples from 19 outpatient adults, eight (42%) had 4-fold increase in anti-G stem antibody response. In both populations, the increase in the mean±SD (reciprocal log₂) anti-G stem titers after RSV infection was statistically significant (hospitalized: 8.3±1.8→10.1±2.8; outpatient: 8.1±1.5→9.9±1.6; for both, p<0.005 by Wilcoxon sign-rank test; Table 1). These data demostrate that: 1) an acute rise in anti-G stem titers is found in ˜40% of RSV subtype A-infected adults; and 2) such titer increases are observed with no obvious correlation to the severity of illness as defined by the initial evaluation in hospitalized vs. outpatient settings.

TABLE 1
Anti-G Stem (aa 151-162; RGH strain) Titers in Acute and
Convalescent Sera from RSV Subtype A-Infected Adults

| Hospitalized | | Outpatient | |
|---|---|---|---|
| 14/32 (44%): ≧4-fold increase in | | 8/19 (42%): ≧4-fold increase in | |
| anti-G stem titers | | anti-G stem titers | |
| Acute | Convalescent | Acute | Convalescent |
| 8.3 ± 1.8 | 10.1 ± 2.8 | 8.1 ± 1.5 | 9.9 ± 1.6 |

Expressed as mean ± SD reciprocal log₂ titers.
For each subject group, the difference between the average convalescent vs. acute anti-GST G stem titers was p < 0.005 by Wilcoxon sign-rank test.

### Discussion of Examples 1-2

A critical component of mAb characterization is the identification of its epitope within the target protein. For L9, a genetic approach was previously utilized in which selection for L9-resistant RSV strains suggested the existence of the cognate epitope within the central unglycosylated region of the RSV G protein (Walsh et al., “Monoclonal Antibody Neutralization Escape Mutants of Respiratory Syncytial Virus With Unique Alterations in the Attachment (G) Protein,” J Gen Virol 79 (Pt 3):479-487 (1998), which is hereby incorporated by reference in its entirety). Examples 1 and 2 now provide biochemical evidence demonstrating that the epitopes of L9 as well as that for K6 are located within the stem region of the RSV G protein, thereby verifying the genetics-based prediction regarding the location of the L9 epitope.

The G stem region has unique structural features. Its primary structure contains the majority of the canonical amino acid sequence HFEVFNFVPCSIC (residues 164-176 of SEQ ID NO:1; shown in FIG. 1C) that is found in all RSV isolates (Johnson et al., “The G Glycoprotein of Human Respiratory Syncytial Viruses of Subgroups A and B: Extensive Sequence Divergence Between Antigenically Related Proteins,” Proc Natl Acad Sci U.S.A. 84:5625-5629 (1987) and Wertz et al., “Nucleotide Sequence of the G Protein Gene of Human Respiratory Syncytial Virus Reveals an Unusual Type of Viral Membrane Protein,” Proc Natl Acad Sci U.S.A. 82:4075-4079 (1985), which are hereby incorporated by reference in their entirety). Overlapping these residues and also present within the G stem is the motif Y/F XFXXFXF (SEQ ID NO:42) (residues 163-170 of SEQ ID NO:1); within this sequence, F163, F165, F168, and F170 are found in G proteins from subtype A strains (e.g., A2, RGH) while Y163 is noted in the G protein from the B1 strain (FIG. 1C). This amino acid motif is also conserved in RSV-related viruses of different host specificities (e.g., ovine and bovine RSV), indicating an evolutionarily conserved structural and/or immunological function (Langedijk et al., “Antigenic Structure of the Central Conserved Region of Protein G of Bovine Respiratory Syncytial Virus,” J Virol 71:4055-4061 (1997) and Langedijk et al., “Type-specific Serologic Diagnosis of Respiratory Syncytial Virus Infection, Based on a Synthetic Peptide of the Attachment Protein G,” J Immunol Methods 193:157-166 (1996), which are hereby incorporated by reference in their entirety).

Of intriguing relevance to these results is the in-solution NMR-based secondary structure model of RSV A2 G-derived residues 149-177, and the antiviral effects of peptides derived from this region (Gorman et al., “Antiviral Activity and Structural Characteristics of the Nonglycosylated Central Subdomain of Human Respiratory Syncytial Virus Attachment (G) Glycoprotein,” J Biol Chem 276:38988-38994 (2001), which is hereby incorporated by reference in its entirety). Due to hydrophobic interactions involving F165, F168, F170, V171, P155, and P156, amino acids 149-177 of the RSV G central unglycosylated region likely forms a disc-like structure with two hydrophobic faces. Interestingly, peptides derived from the central unglycosylated region and bearing residues EVFNF (i.e., amino acid residues 166-170 of SEQ ID NO:1) reduced RSV-associated cytopathic effects in infected HEp-2 cells. Based on these and other observations, several possible roles for residues 166-170 have been proposed, including a self-association domain among RSV G monomers and/or interactions with a cellular RSV G protein receptor (Gorman et al., “Antiviral Activity and Structural Characteristics of the Nonglycosylated Central Subdomain of Human Respiratory Syncytial Virus Attachment (G) Glycoprotein,” J Biol Chem 276:38988-38994 (2001), which is hereby incorporated by reference in its entirety). In either scenario, the peptide would then function as a “dominant negative” to alter the structure and/or function of the RSV G protein. The results described herein indicate that somewhat unexpectedly, ≧1 neutralizing epitope is found within and flanking RSV G residues 166-170. Furthermore, the epitope mapping studies raise the possibility that L9 and other mAbs recognizing the G stem-embedded epitopes may directly or indirectly affect RSV G structure (e.g., destabilize multimerization) and/or function (i.e., block interactions with the host target cell). Previously the isolation of an L9-resistant virus that bore mutations outside of the G stem region was reported; perhaps these mutations represent second site/compensatory changes that counteract the action(s) of L9 mAb on RSV G structure/function (Walsh et al., “Monoclonal Antibody Neutralization Escape Mutants of Respiratory Syncytial Virus With Unique Alterations in the Attachment (G) Protein,” J Gen Virol 79 (Pt 3):479-487 (1998), which is hereby incorporated by reference in its entirety).

Within the RSV G protein, short “protective” B-cell epitopes have been defined by Plotnicky-Gilquin et al., “Identification of Multiple Protective Epitopes (Protectopes) in the Central Conserved Domain of a Prototype Human Respiratory Syncytial Virus G Protein,” J Virol 73:5637-5645 (1999), which is hereby incorporated by reference in its entirety. Mice were injected with bacterially derived BBG2Na (amino acid residues 130-230 from the RSV Long strain G protein conjugated to the albumin-binding domain of streptococcal protein G) and the resulting mAbs were shown to be non-RSV neutralizing but protective against intranasal RSV challenge. Overlapping peptides spanning the RSV G moiety within BBG2Na were then used to demonstrate that these mAbs bound to short epitopes (amino acid residues 152-163, 165-172, 171-187, and 194-206) within RSV G; two of these epitopes are located within the GST-G stem region used in the Examples presented herein (Plotnicky-Gilquin et al., “Identification of Multiple Protective Epitopes (Protectopes) in the Central Conserved Domain of a Prototype Human Respiratory Syncytial Virus G Protein,” J Virol 73:5637-5645 (1999), which is hereby incorporated by reference in its entirety). It should be noted that L9 and K6 are both neutralizing and subtype-independent, whereas none of the protective epitope-recognizing mAbs were neutralizing, and it is unclear whether the protective effect against viral challenge was subtype-independent. These differences in the functional profiles of the various mAbs may be due to the immunogen (purified, native RSV G protein from RSV infected mammalian cells vs. bacterially derived, refolded partial RSV G protein) used to generate the respective mAbs.

Despite the key immunological activities that have been associated with RSV G protein, the serological characterization of RSV G epitopes, especially those within the G stem region, in the context of human RSV infections remains incomplete. A very limited number of adult human sera (n=2) were used to study the reactogenicity of BBG2Na-associated protective epitopes (Plotnicky-Gilquin et al., “Identification of Multiple Protective Epitopes (Protectopes) in the Central Conserved Domain of a Prototype Human Respiratory Syncytial Virus G Protein,” J Virol 73:5637-5645 (1999), which is hereby incorporated by reference in its entirety). Other RSV G-based human serological screening studies utilized bacterially synthesized, genetically hypervariable regions flanking the central unglycosylated region or G-derived peptides (overlapping or non-overlapping) to screen adult or pediatric sera (Cane, P. A., “Analysis of Linear Epitopes Recognized by the Primary Human Antibody Response to a Variable Region of the Attachment (G) Protein of Respiratory Syncytial Virus,” J Med Virol 51:297-304 (1997); Cane et al., “Analysis of the Human Serological Immune Response to a Variable Region of the Attachment (G) Protein of Respiratory Syncytial Virus During Primary Infection,” J Med Virol 48:253-261 (1996); Cane et al., “Analysis of the Human Serological Immune Response to a Variable Region of the Attachment (G) Protein of Respiratory Syncytial Virus During Primary Infection,” J Med Virol 48:253-261 (1996); Palomo et al., “Evaluation of the Antibody Specificities of Human Convalescent-Phase Sera Against the Attachment (G) Protein of Human Respiratory Syncytial Virus: Influence of Strain Variation and Carbohydrate Side Chains,” J Med Virol 60:468-474 (2000); and Shinoff et al., “Young Infants Can Develop Protective Levels of Neutralizing Antibody After Infection With Respiratory Syncytial Virus,” J Infect Dis 198:1007-1015 (2008), which are hereby incorporated by reference in their entirety). The Examples presented herein provide serological evidence indicating the G stem region bears ≧1 neutralizing epitope and that a ≧4-fold increase in serum reactogenicity against this region is noted in a significant proportion of RSV subtype A-infected adults. These data demonstrate that the G stem region is immunologically significant in human RSV infections. Although the G stem region is highly conserved among the A2, B1, and RGH strains (FIG. 1C), the human serum reactogenicity against the G stem from the clinical isolate (RGH strain) in ELISAs was somewhat less than that against the G stem of the laboratory-derived A2 and B1 strains (FIG. 4); this may illustrate that very subtle amino acid changes within the stem affect mAb-epitope interactions in vitro. The human serological studies will be extended to determine the reactogenicity against other regions of the RSV G central domain among RSV-infected adults. Interestingly, the conserved nature of G stem residues among clinical isolates (FIG. 1C) and the mapping of subtype-independent neutralizing epitopes in the Examples described herein support the belief that the G stem region is a target of prophylactic and/or therapeutic agents for RSV infection. As described infra, the G stem region may be engineered as epitopes onto RSV preclinical vaccine candidates.

### Example 3

### Design and Characterization of RSV-G Self-Folding Polypeptides

Three full-length RSV G amino acid sequences used for analysis include those from the RSV Long and B1 strains and that from a recent clinical isolate (RGH strain; genotype A5, isolated in 1999). BLAST and extensive CD (conserved domain) searches revealed no significant structural homologies except to the pneumovirus G protein family. Also, an order/disorder analysis of the RSV G protein amino acid sequences were performed (using DisEMBL). As expected, an ordered structure around the amino terminus and mapping to the predicted TM domain was identified. However, of the three analyses provided on the server, two showed the following amino acids sequence corresponding to amino acids 151-200 from RGH G sequence (SEQ ID NO:1). The disordered portion is shown in bold.

RQNKPPNKPN DDFHFEVFNF VPCSICSNNP TCWAICKRIP SKKPGKKTTT
RQNKPPNKPN DDFHFEVFNF VPCSICSNNP TCWAICKRIP SKKPGKKTTT

These results are significant for three reasons. First, they are consistent with previous observations that the four central cysteines likely exist in an ordered loop structure and that the region containing the conserved amino acid EVFNF motif (amino acid residues 166-170 of SEQ ID NO:1) also exists in a loop-like configuration in solution; thus, recombinant expression of these amino acid sequences may result in self-folding subdomains. Second, these amino acid sequences bear most of the previously identified B-cell epitopes that were defined in a bacterially expressed fragment of RSV G in BBG2Na protein. Lastly, the unglycosylated region of RSV G is highly conserved among RSV G amino acid sequences and antibodies generated against this region should have a greater probability of cross-neutralizing a broad spectrum of clinical RSV isolates.

Based on the above analysis, the following RSV RGH strain-derived amino acid sequences, or portions thereof, will be expressed in recombinant systems (prokaryotic, including E. coli, and eukaryotic, including but not limited to insect, mammalian, and yeast cells).

The entire putative unglycosylated region corresponding to amino acids 151-190 of the RSV RGH strain (SEQ ID NO:1):

(SEQ ID NO: 3)
RQNKPPNKPN DDFHFEVFNF VPCSICSNNP TCWAICKRIP.

The amino acid sequences flanking the looped structures are included to facilitate the solubility of the resulting protein, especially in the presence of hydrophobic amino acid side chains within the EVFNF motif.

The amino terminal sequence from SEQ ID NO:3, extending up to the fourth cysteine (expected to bear the cysteine loop structure), corresponding to amino acids 151-186 of the RSV RGH strain (SEQ ID NO:1):

(SEQ ID NO: 4)
RQNKPPNKPN DDFHFEVFNF VPCSICSNNP TCWAIC.

The unglycosylated region extending up to the first cysteine, corresponding to amino acids 151-172 of SEQ ID NO:1:

RQNKPPNKPN DDFHFEVFNF VP (SEQ ID NO: 5)

Following expression and purification in recombinant systems, the relevant RSV G subdomains will be utilized as an immunogen in the context of highly immunogenic adjuvants, including but not limited to bacterially derived flagellin or in the context of human papillomavirus (HPV) L1 virus-like particles (VLPs). Fusion protein construction may necessitate engineering of short amino-CGG or -GGC carboxy-terminus linker sequences and physical cross-linking using a hetero-bifunctional cross-linking agent as described in WO/2009/055491 to Murata et al., which is hereby incorporated by reference in its entirety. Using an existing panel of monoclonal anti-RSV G antibodies and polyclonal anti-RSV G antibodies, the recombinant proteins bearing RSV G-derived amino acid sequences will be characterized using ELISAs, immunoblots, and immunoprecipitation experiments.

### Example 4

### Design and Characterization of RSV-F Self-Folding Polypeptides

The RSV F amino acid sequence from RSV RGH strain (SEQ ID NO:2) was subjected to a order/disorder search (DisEMBL), which revealed the following:

1. Disordered by Loops/coils definition:

>RSV F_LOOPS 1-8, 63-73, 110-131, 196-215, 237-248, 261-281,
304-403, 417-450, 459-490, 512-525, 550-574
MELPILKTNA ITTILAAVTL CFASSQNITE EFYQSTCSAV SKGYLSALRT GWYTSVITIE
LSNIKENKCN GTDAKVKLIK QELDKYKNAV TELQLLMQST PAANNRARRE LPRFMNYTLN
NTKNNNVTLS KKRKRRFLGF LLGVGSAIAS GIAVSKVLHL EGEVNKIKSA LLSTNKAVVS
LSNGVSVLTS KVLDLKNYID KQLLPIVNKQ SCSISNIETV IEFQQKNNRL LEITREFSVN
AGVTTPVSTY MLTNSELLSL INDMPITNDQ KKLMSNNVQI VRQQSYSIMS IIKEEVLAYV
VQLPLYGVID TPCWKLHTSP LCTTNTKEGS NICLTRTDRG WYCDNAGSVS FFPQAETCKV
QSNRVFCDTM NSLTLPSEVN LCNIDIFNPK YDCKIMTSKA DVSSSVITSL GAIVSCYGKT
KCTASNKNRG IIKTFSNGCD YVSNKGVDTV SVGNTLYYVN KQEGKSLYVK GEPIINFYDP
LVFPSDEFDA SISQVNEKIN QSLAFIRKSD ELLHNVNVGK STTNIMITTI IIVIIVILLL
LIAVGLFLYC KARSTPVTLS KDQLSGINNI AFSN

2. Disordered by Hot-loops definition:

>RSV F_HOTLOOPS 61-72, 114-133, 237-247, 318-331, 419-446,
518-526, 555-574
MELPILKTNA ITTILAAVTL CFASSQNITE EFYQSTCSAV SKGYLSALRT GWYTSVITIE
LSNIKENKCN GTDAKVKLIK QELDKYKNAV TELQLLMQST PAANNRARRE LPRFMNYTLN
NTKNNNVTLS KKRKRRFLGF LLGVGSAIAS GIAVSKVLHL EGEVNKIKSA LLSTNKAVVS
LSNGVSVLTS KVLDLKNYID KQLLPIVNKQ SCSISNIETV IEFQQKNNRL LEITREFSVN
AGVTTPVSTY MLTNSELLSL INDMPITNDQ KKLMSNNVQI VRQQSYSIMS IIKEEVLAYV
VQLPLYGVID TPCWKLHTSP LCTTNTKEGS NICLTRTDRG WYCDNAGSVS FFPQAETCKV
QSNRVFCDTM NSLTLPSEVN LCNIDIFNPK YDCKIMTSKA DVSSSVITSL GAIVSCYGKT
KCTASNKNRG IIKTFSNGCD YVSNKGVDTV SVGNTLYYVN KQEGKSLYVK GEPIINFYDP
LVFPSDEFDA SISQVNEKIN QSLAFIRKSD ELLHNVNVGK STTNIMITTI IIVIIVILLL
LIAVGLFLYC KARSTPVTLS KDQLSGINNI AFSN

3. Disordered by Remark-465 definition:

>RSV F_REM465 none
MELPILKTNA ITTILAAVTL CFASSQNITE EFYQSTCSAV SKGYLSALRT GWYTSVITIE
LSNIKENKCN GTDAKVKLIK QELDKYKNAV TELQLLMQST PAANNRARRE LPRFMNYTLN
NTKNNNVTLS KKRKRRFLGF LLGVGSAIAS GIAVSKVLHL EGEVNKIKSA LLSTNKAVVS
LSNGVSVLTS KVLDLKNYID KQLLPIVNKQ SCSISNIETV IEFQQKNNRL LEITREFSVN
AGVTTPVSTY MLTNSELLSL INDMPITNDQ KKLMSNNVQI VRQQSYSIMS IIKEEVLAYV
VQLPLYGVID TPCWKLHTSP LCTTNTKEGS NICLTRTDRG WYCDNAGSVS FFPQAETCKV
QSNRVFCDTM NSLTLPSEVN LCNIDIFNPK YDCKIMTSKA DVSSSVITSL GAIVSCYGKT
KCTASNKNRG IIKTFSNGCD YVSNKGVDTV SVGNTLYYVN KQEGKSLYVK GEPIINFYDP
LVFPSDEFDA SISQVNEKIN QSLAFIRKSD ELLHNVNVGK STTNIMITTI IIVIIVILLL
LIAVGLFLYC KARSTPVTLS KDQLSGINNI AFSN

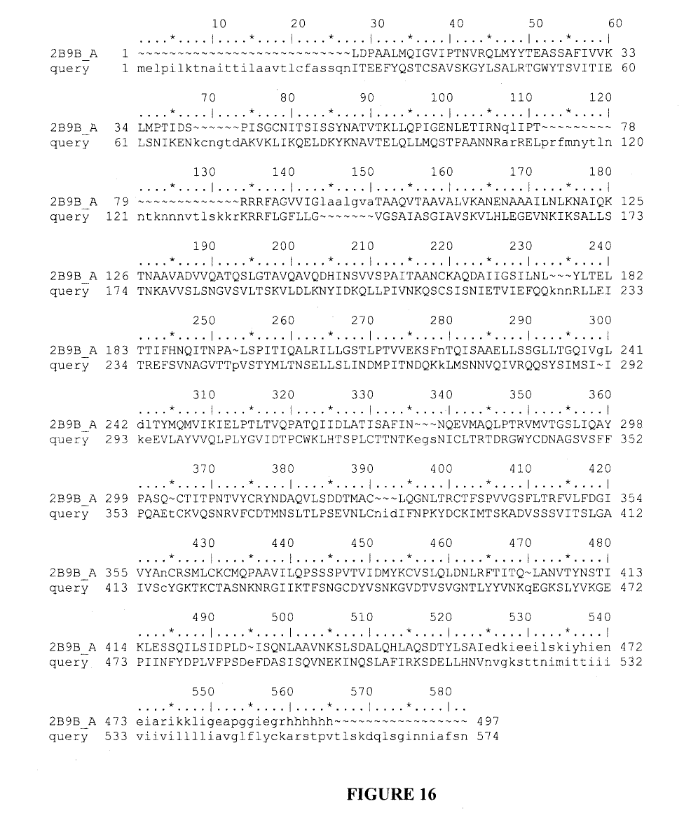

These analyses confirm that, in general and with some regional/analysis-specific exceptions, RSV F likely possesses a fairly ordered structure. Accordingly, the RSV F amino acid sequence from RSV RGH strain was used in a BLAST/CDD search to identify amino acid homologies. The most significant amino acid match with an existing structure was with 2B9B (Protein Data Base accession number for the pre-fusion conformation of PIV5 F) (see RSV F monomer to PIV5 F monomer amino acid sequence alignment in FIG. 16). This sequence match, as well as with the NDV F-derived model of RSV F as described by Smith et al., “Modeling the Structure of the Fusion Protein from Human Respiratory Syncytial Virus,” Protein Eng 15:365-371 (2003), which is hereby incorporated by reference in its entirety, were used for the following predictions.

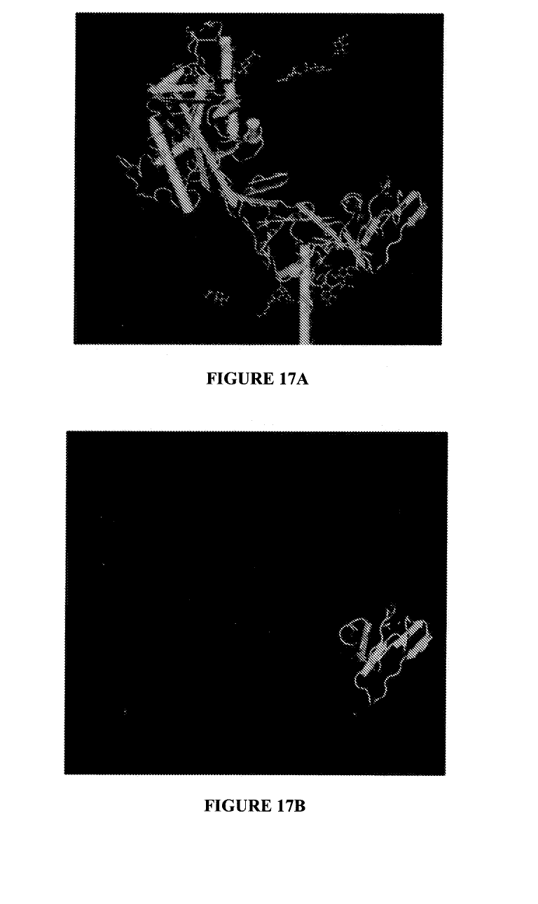

The RSV antigenic domain IV/V/VI (including aa 423-436) maps to amino acids 365-378 within a surface-exposed region of structural domain II of PIV5 F protein (see FIG. 17A; highlighted amino acid sequence shown in light gray in ribbon diagram). The entire structural domain II of PIV 5/NDV is expected to fold independently and without significant changes in the structure between the prefusion and post-fusion states. The PIV5 structural domain II, alone, is shown in ribbon diagram of FIG. 17B.

Within the RSV F protein of the RGH strain, the homologous amino acid sequence to structural domain II of NDV/PIV5 F is amino acids 382-459:

(SEQ ID NO: 7)
CNIDIFNPKYDCKIMTSKADVSSSVITSLGAIVSCYGKTKCTASNKNRG
IIKTFSNGCDYVSNKGVDTVSVGNTLYYV

Within this sequence, cysteine residues are noted at positions 1, 12, 35, 41 and 58 (corresponding to amino acid positions 382, 393, 416, 422, and 439 of SEQ ID NO:2). Analysis of the cysteine disulfide bonds within RSV F is illustrated in FIG. 18. Thus, it is reasonable to assume that if the structural domain II of RSV F is a thermodynamically stable, self-folding structure, then the disulfide bonds between C382-393 and 416-422 should form as expected. This model also predicts a disulfide bond between C37 and C439. To address this issue, versions of RSV F structural domain II bearing a cysteine to serine substitution at amino acid position 58 (or 439 of SEQ ID NO:1) will be generated so as to increase domain II self-folding.

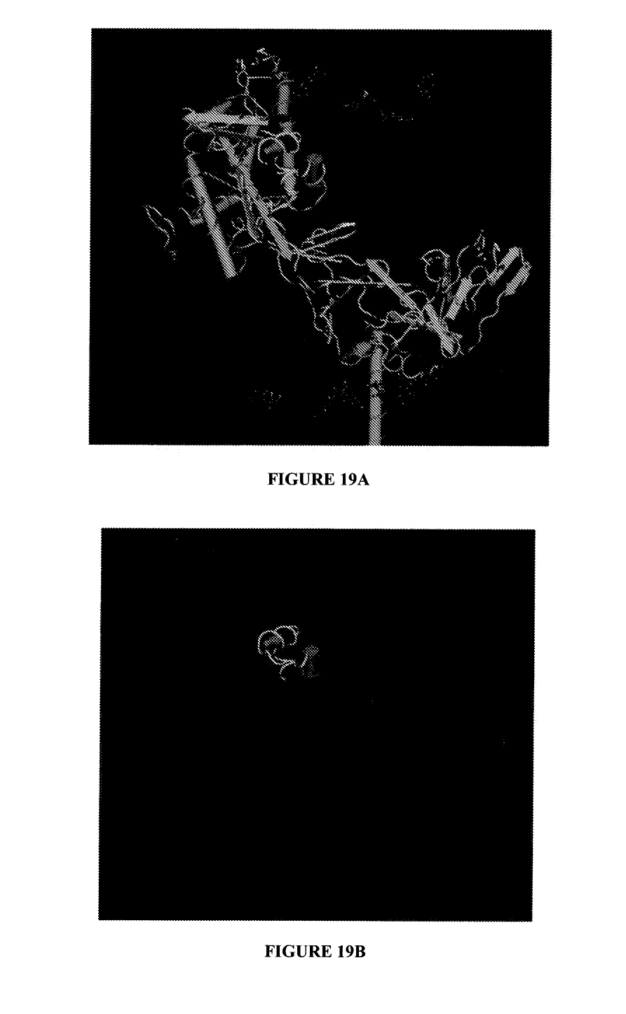

The RSV F antigenic domain II (amino acid residues 255-278) has homology to amino acids 203-227 within structural domain III of PIV5 F. FIG. 19A is a ribbon diagram showing the homologous amino acid portion of the RSV antigenic domain II in the context of the entire PIV 5 F monomer (highlighted in light gray) and FIG. 19B shows this homologous region alone.

Within RSV F, the amino acid sequence homologous to structural domain III of PIV5 F is predicted to be either amino acids 233-303 of SEQ ID NO:2 (encompassing N1-N4 portions as well as structural domain III beta sheets b and c):

(SEQ ID NO: 8)
ITREFSVNAGVTTPVSTYMLTNSELLSLINDMPITNDQKKLMSNNVQIV
RQQSYSIMSIIKEEVLAYVVQL

or amino acids 233-286 of SEQ ID NO:2 (encompassing only the N1-N4 portions of structural domain III):

(SEQ ID NO: 9)
ITREFSVNAGVTTPVSTYMLTNSELLSLINDMPITNDQKKLMSNNVQIV
RQQST

Neither of these two sequences contain cysteines.

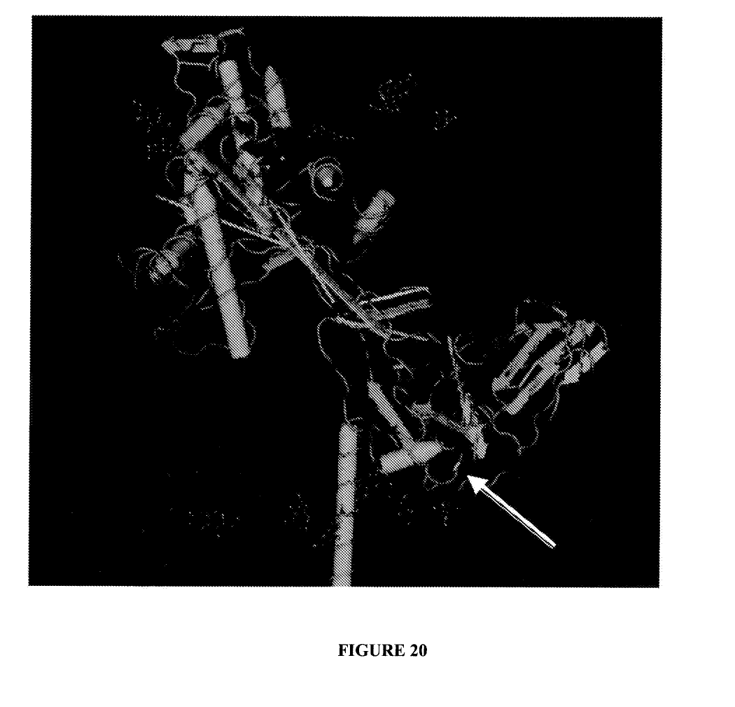

The RSV antigenic domain I, including amino acid 389, corresponds to amino acid 328 within PIV5 F. This amino acid position maps to a surface-exposed region between helix 2 and structural domain II of PIV5 F protein (FIG. 20). To facilitate the folding of the amino acid 389-bearing RSV F domain, amino acid 389 will be included in a larger context bearing both helix 2 and structural domain II-analogous portions of RSV F (i.e., the polypeptide comprising amino acids 374-459):

(SEQ ID NO: 6)
TLPSEVNLCNIDIFNPKYDCKIMTSKADVSSSVITSLGAIVSCYGKTKC
TASNKNRGIIKTFSNGCDYVSNKGVDTVSVGNTLYYV

As previously noted, to avoid the presence of an uncoupled cysteine that may form spurious, unintended disulfide bonds, derivatives of the above amino acid sequence bearing a cysteine to serine substitution at position 66 of SEQ ID NO:6 will be generated.

These F derivative polypeptides will be expressed and characterized as previously described for RSV G derivative polypeptides, except that the relevant panel of anti-RSV F monoclonal and polyclonal antibodies will be used in appropriate experiments. In the case of RSV F subdomains involving structural domain III homologous regions, it is not known a priori whether the resulting RSV F derivative will exist primarily in a pre-fusion or post-fusion state, the latter bearing significantly altered conformations. Thus, the use of these anti-F antibodies will prove useful in characterizing the resulting recombinant proteins.

### Example 5

### Characterization of T-cell Response Following Immunization

In vivo immunization studies will be performed using the above described RSV G and RSV F polypeptides, fusion proteins, immunogenic conjugates, or the nucleic acids encoding the same, to assess their immunogenicity. The sufficiency of the immune response to promote virus neutralization will be assessed via a neutralization assay.

Following immunization, the generation of CD4+(Th1- or Th2-biased) and/or RSV-specific CTL responses accompanying a neutralizing antibody response will be determined. To this end, the following assays will be performed: 1) intracellular cytokine staining (ICS) of splenocytes; 2) ELISA to determine the levels of IL-4, IL-5, and γ-IFN secreted by splenocytes; and 3) fluorescence-based CTL assay.

For each immunization group, splenocytes will be harvested under sterile conditions using standard procedures and a 100 μm cell strainer will be used to generate single-cell splenocytes in PBS/1% fetal calf serum (FCS). The cells will then be resuspended in 5 mL hemolysis buffer (150 mM NH₄Cl, 1 mM KHCO₃, and 0.1 mM EDTA pH. 7.2-7.4) and thereafter washed×3 with wash buffer before resuspension to 1×10⁶ cells/ml in RPMI 1640/10% FCS (Deml et al., “Virus-like Particles: A Novel Tool for the Induction and Monitoring of Both T-helper and Cytotoxic T-lymphocyte Activity,” Methods Mol Med 94:133-157 (2004), which is hereby incorporated by reference in its entirety).

For each immunization group, pooled splenocytes (2×10⁶ cells total) will be placed into 6 ml round-bottom tubes (Falcon) in duplicate. To ensure robust measurement of ICS signal, one set will be incubated for 2 hrs and the other will be incubated for 10 hours at 37° C. with UV-inactivated RGH strain RSV (10⁶-10⁷ pfu/ml) or media alone (Jackson et al., “Different Patterns of Cytokine Induction in Cultures of Respiratory Syncytial (RS) Virus-specific Human TH Cell Lines Following Stimulation with RS Virus and RS Virus Proteins,” J Med Virol 49:161-169 (1996), which is hereby incorporated by reference in its entirety). Each sample will then be supplemented with 1 μl monensin (GolgiStop; BD) per tube for additional 6 hrs; based on this strategy, two time points will be obtained, one at 8 hrs and the other at 16 hrs, for each spleen sample/ICS. The cells will then be washed once in PBS/2% FCS and surface stained with either Quantum Red-conjugated rat α-mouse-CD4 or CD8 mAb (Sigma) for 30 minutes at 4° C. Cells will then be washed, fixed, and permeabilized (Cytofix/Cytoperm; BD), and intracellularly stained using a commercially available kit (BD) with phycoerythrin-conjugated rat α-mouse IFN-γ antibody and rat a-mouse anti-IL-4-FITC antibody (BD). Cells will be analyzed using a 3-color FACscanner flow cytometer (BD FACScan) and CellQuest software (BD) (Deml et al., “Virus-like Particles: A Novel Tool for the Induction and Monitoring of Both T-helper and Cytotoxic T-lymphocyte Activity,” Methods Mol Med 94:133-157 (2004); Fischer et al., “Pertussis Toxin Sensitization Alters the Pathogenesis of Subsequent Respiratory Syncytial Virus Infection,” J Infect Dis 182:1029-1038 (2000); Rutigliano et al., “Treatment with anti-LFA-1 Delays the CD8+Cytotoxic-T-Lymphocyte Response and Viral Clearance in Mice with Primary Respiratory Syncytial Virus Infection,” J Virol 78:3014-3023 (2004); Pala et al., “Flow Cytometric Measurement of Intracellular Cytokines,” J Immunol Methods 243:107-124 (2000), each of which is hereby incorporated by reference in its entirety).

For each immunization group, pooled spleens cells will be plated in triplicate into 96 well round bottom plates (2×10⁵ cells in 100 μl/well). The cells will be stimulated with UV-inactivated RGH strain RSV (10⁶ pfu/ml), purified RSV A2 F protein (100 ng/ml), phytohemagglutinin (PHA; Sigma-Aldrich) at 10 μg/ml, or media alone. Cells will be incubated at 37° C. with 5% CO₂ for 48 hours. Supernatants from each well will be harvested and assayed for secreted IFN-γ, IL-4, and IL-5 using an ELISA-based commercially available kit (BD) (Fischer et al., “Pertussis Toxin Sensitization Alters the Pathogenesis of Subsequent Respiratory Syncytial Virus Infection,” J Infect Dis 182:1029-1038 (2000); Rutigliano et al., “Treatment with anti-LFA-1 Delays the CD8+Cytotoxic-T-lymphocyte Response and Viral Clearance in Mice with Primary Respiratory Syncytial Virus Infection,” J Virol 78:3014-3023 (2004), each of which is hereby incorporated by reference in its entirety).

For effector cells, splenocytes from each immunization group will be cultured in T25 flasks at 10⁶ cells/mL in RPMI/10% FCS. Each flask of cells will be stimulated ex vivo by addition of 10⁷ pfu of live RGH strain RSV and cultured at 37° C. and 5% CO₂ for 5 days. For target cells, BCH4 cells (derived from BAL/c embryo fibroblasts persistently infected with the Long strain of RSV) and B4 cells (a BALB/c fibroblast cell line uninfected with RSV) will be labeled (Fernie et al., “The Development of Balb/c Cells Persistently Infected with Respiratory Syncytial Virus: Presence of Ribonucleoprotein on the Cell Surface,” Proc Soc Exp Biol Med 167:83-86 (1981), which is hereby incorporated by reference in its entirety. BCH4 cells will be labeled with 5 μM 5-(and -6)-carboxyfluorescein diacetate, succinimidyl ester (CFSE) and B4 cells will be labeled with 0.5 μM CFSE. The labeled cells will then be washed with RPMI/10% FCS and plated onto 96 well plates (Nunc) at 20,000 cells/well in 100 μl media. Equal numbers (10,000 cells) of CFSE high and CFSE low target cells will be incubated simultaneously with the effector cells and incubated for 2-4 hrs at 37° C. The cells will then be analyzed by flow cytometry, and the percentage of RSV-specific target cell lysis will be calculated as 100−(% CFSE high cells/% CFSE low cells) (Rutigliano et al., “Identification of an H-2D(b)-restricted CD8+Cytotoxic T Lymphocyte Epitope in the Matrix Protein of Respiratory Syncytial Virus,” Virology 337:335-343 (2005), which is hereby incorporated by reference in its entirety).

### Example 6

### Protective Efficacy of RSV G and RSV F Self-Folding Neutralizing Epitope Bearing Polypeptides on RSV Challenge

As a final assessment of RSV G and F polypeptide immunogenic efficacy, mouse protection studies will be performed. First, the virus replication pattern for the RGH wild type RSV strain will be established. Mice (n=24) will be anesthetized with Ketamine (60-90 mg/kg) IP plus Xylazine (4-8 mg/kg) IP or acepromazine (1-2 mg/kg IP) and then inoculated intranasally with 10⁶ pfu RGH RSV in 50 uL total volume of MEM/5% FCS from RSV-infected HEp-2 cells. On days 2, 3, 4, 5, 7, and 10, four mice will be weighed, sacrificed and subjected to bronchoalveolar lavage (BAL) and nasal wash (NW) using a 19-gauge blunt-end needle to inject ˜0.5 ml PBS/5% FCS into the trachea or nares (Walsh et al., “Protection from Respiratory Syncytial Virus Infection in Cotton Rats by Passive Transfer of Monoclonal Antibodies,” Infect Immun 43:756-758 (1984); Graham et al., “Primary Respiratory Syncytial Virus Infection in Mice,” J Med Virol 26:153-162 (1988), each of which is hereby incorporated by reference in its entirety). The samples will be centrifuged and virus titer determined by plaque assays using HEp-2 cells. The weights and plaque assay data will be plotted to determine the clinical manifestation of RSV infection and the kinetics of virus replication, respectively (Graham et al., “Primary Respiratory Syncytial Virus Infection in Mice,” J Med Virol 26:153-162 (1988), which is hereby incorporated by reference in its entirety).

Mice (6/group) will undergo two vaccinations (d0 and d14) with each RSV G and F polypeptides that demonstrated an immune response that was effective for in vitro neutralization studies. Any modifications, such as use of adjuvant or altered amount of the polypeptide in each injection, that were found to optimize immunogenicity of the polypeptides will also be used for this analysis. Negative and positive control mice will receive PBS or live RGH strain RSV, respectively. Four weeks (d42) later, mice will be challenged intranasally with 10⁶ pfu RGH RSV strain. When peak RSV viral titers are expected (as determined by viral kinetic experiments noted above), the mice will be sacrificed for BAL, and both NW and RSV titers will be measured as described above. As a qualitative measure of the severity of RSV infection, each animal will be weighed daily until sacrifice. Degree of protection by each of the RSV G and F polypeptides will be determined by comparison of the weights and viral titers to those of the negative control group. The initial choice of number of mice to be used is based on the expected minimum differences in viral titers in the immunized vs. non-immunized groups using the Student's t test. If deemed necessary based on the parametric/non-parametric distribution of data points, the Mann-Whitney rank sum test will also be used.

### Example 7

### Characterization of the Neutralizing Antibody Response Induced by the RSV G and RSV F Self-Folding Neutralizing Epitope Bearing Polypeptides

A standard plaque reduction neutralization assay will be performed using pre- and post-immunization serum samples. Sera will be serially diluted starting at 1:25 in MEM/5% FCS. Each serum dilution (300 μL) will be mixed with 300 μL of MEM containing 215 plaque forming units (pfu) of RSV and incubated at RT for 30 min. An aliquot (200 μl) of each mixture will then be inoculated onto preset HEp-2 monolayers in 24 well plates (Costar) for 2 hrs at RT. The inoculum will be removed and the monolayer overlayed with 2 ml of 0.5% methylcellulose in MEM/5% FCS and incubated for 4 days at 37° C. Plates will be fixed with 1 ml of 0.5% glutaraldehyde/PBS, washed, and stained with methylene blue. RSV plaques will be visualized and counted using a dissecting microscope. The neutralization titer is defined as the dilution (expressed as log₂ dilution) resulting in 50% plaque reduction compared to control wells containing virus without serum (Murphy et al., “Dissociation Between Serum Neutralizing and Glycoprotein Antibody Responses of Infants and Children Who Received Inactivated Respiratory Syncytial Virus Vaccine,” J Clin Microbiol 24:197-202 (1986); Falsey et al., “Serologic Evidence of Respiratory Syncytial Virus Infection in Nursing Home Patients,” J Infect Dis 162:568-569 (1990); Falsey et al., “Humoral Immunity to Respiratory Syncytial Virus Infection in the Elderly,” J Med Virol 36:39-43 (1992), each of which is hereby incorporated by reference in its entirety).

A standard fusion inhibition assay will be performed using pre- and post-immunization serum samples. Monolayers of HEp-2 cells will be infected with 1,000 pfu of RSV and at eight hours post-infection, incubated with MEM containing serially diluted mouse sera starting at 1:25 in MEM/5% FCS. Following two days of incubation at 37° C., the cell monolayers will be washed, fixed with acetone, and virus-infected cells will be visualized by indirect immunofluorescence microscopy and using fluorescein isothiocyanate (FITC)-conjugated goat anti-mouse monoclonal antibody and a mouse monoclonal antibody to RSV nucleocapsid protein. Appropriate controls will be used, including a fusion-inhibiting monoclonal antibody to RSV F protein and preimmune mouse serum. The ability of each serum sample to inhibit syncytium formation and new foci of infection will be assayed (Walsh, et al., “Purification and Characterization of the Respiratory Syncytial Virus Fusion Protein,” J. Gen Virol. 66: 409-415 (1985), which is hereby incorporated by reference in its entirety).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

It is expressly intended that all combinations of those elements and/or method steps which perform substantially the same function in substantially the same way to achieve the same results are within the scope of the invention. Moreover, it should be recognized that elements and/or method steps shown and/or described in connection with any disclosed form or embodiment of the invention may be incorporated in any other disclosed or described or suggested form or embodiment.

1. An isolated polypeptide comprising a polypeptide fragment of a respiratory syncytial virus (RSV) attachment glycoprotein (G) protein or fusion (F) protein, wherein the polypeptide fragment is a self-folding, soluble, stable fragment that lacks N- or O-glycosylation sites and comprises a neutralizing epitope.

2. The isolated polypeptide according to claim 1, wherein the polypeptide fragment comprises the amino acid sequence of SEQ ID NO:3, SEQ ID NO:4, or SEQ ID NO:5.

3. (canceled)

4. The isolated polypeptide according to claim 1, wherein the polypeptide fragment comprises the amino acid sequence of SEQ ID NO:6, SEQ ID NO:7, SEQ ID NO:8, or SEQ ID NO:9.

5. (canceled)

6. The isolated polypeptide according to claim 1, wherein the polypeptide fragment comprises at least two cysteine residues.

7. (canceled)

8. The isolated polypeptide according to claim 1, wherein the polypeptide fragment is less than 90 amino acids in length.

9. The isolated polypeptide according to claim 8, wherein the polypeptide fragment is greater than 20 amino acids in length.

10. A fusion protein comprising the polypeptide fragment according to claim 1 linked by an in-frame fusion to an adjuvant polypeptide.

11. The fusion protein according to claim 10, wherein the adjuvant polypeptide is selected from the group consisting of flagellin, human papillomavirus L1 or L2 protein, herpes simplex glycoprotein D (gD), complement C4 binding protein, TLR-4 ligand, and IL-1β.

12. The fusion protein according to claim 10, further comprising a linker sequence positioned between the polypeptide fragment and the adjuvant polypeptide.

13. (canceled)

14. An immunogenic conjugate comprising the polypeptide fragment according to claim 1 conjugated to an immunogenic carrier molecule.

15. The immunogenic conjugate according to claim 14, wherein the immunogenic carrier molecule is covalently or non-covalently bonded to the polypeptide fragment.

16. (canceled)

17. An isolated polynucleotide encoding a polypeptide according to claim 1.

18. The isolated polynucleotide according to claim 17, wherein the polynucleotide comprises any one of the nucleic acid sequences selected from the group consisting of SEQ ID NO:10, SEQ ID NO:11, SEQ ID NO:12, SEQ ID NO:13, SEQ ID NO:14, and SEQ ID NO:15.

19. The isolated polynucleotide according to claim 17, wherein the polynucleotide comprises any one of the nucleic acid sequences selected from the group consisting of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, and SEQ ID NO:33.

20. The isolated polynucleotide according to claim 17, wherein the polynucleotide is codon-optimized for expression of the polypeptide in a eukaryotic host cell or yeast.

21. A recombinant transgene comprising a polynucleotide according to claim 17 operably coupled to a promoter-effective DNA molecule, a leader DNA sequence comprising a start-codon, and a transcription termination sequence.

22. A recombinant vector comprising the isolated polynucleotide according to claim 17.

23. The recombinant vector according to claim 22 selected from the group consisting of a bacterial vector, a yeast vector, and a viral vector.

24. (canceled)

25. A host cell comprising a transgene according to claim 21.

26. (canceled)

27. A pharmaceutical composition comprising:
an isolated polypeptide according to claim 1; and
a pharmaceutically acceptable carrier.

28. The pharmaceutical composition according to claim 27 further comprising a distinct adjuvant.

29. The pharmaceutical composition according to claim 28, wherein the distinct adjuvant is selected from the group consisting of flagellin, Freund's complete or incomplete adjuvant, aluminum hydroxide, lysolecithin, pluronic polyols, polyanions, peptides, oil emulsion, dinitrophenol, iscomatrix, and liposome polycation DNA particles.

30. (canceled)

31. The pharmaceutical composition according to claim 27 further comprising a delivery vehicle.

32-33. (canceled)

34. A method of inducing a neutralizing immune response against respiratory syncytial virus (RSV) in a subject comprising:
administering to the subject a pharmaceutical composition according to claim 27 in an amount effective to induce a neutralizing immune response against RSV.

35-38. (canceled)
